(19) United States
(12) Patent Application Publication
(10) Pub. No.: US 2005/0171443 A1
(43) Pub. Date: Aug. 4, 2005

(54) Apparatus and method for non-invasive monitoring of cardiac performance

(76) Inventors: Miguel Gorenberg, Haifa, IL (US); Hector Rotstein, Haifa, IL (US); Michael Naroditzky, Carmiel, IL (US); Alon Marmor, Carmiel, IL (US); Ehud Dafni, Caesaria, IL (US)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 10/492,336

(22) PCT Filed: Sep. 2, 2003
(86) PCT No.: PCT/IL03/00720

(51) Int. Cl. A61B 5/02

(52) U.S. Cl. 600/490

(57) ABSTRACT

A non-invasive apparatus for measuring cardiac mechanical performance of a patient, the apparatus comprising a pressure applying element (301) mountable on a limb of the patient for applying pressure high enough to make a segment of an artery within the limb achieve a collapsed state and empty it from blood at least momentarily; at least one of a plurality of sensors coupled to the pressure applying element, sensing mechanical changes corresponding to volumetric changes in the artery as the artery progressively recuperates from its collapsed state; processing unit (303) communicating with the sensors for receiving output corresponding to the mechanical changes from the sensors and computing factors correlated with blood flow and calculate parameters indicating heart performance.

### FIELD OF THE INVENTION

The present invention relates to non-invasive monitoring of heart mechanical performance. More particularly, the present invention is related to a noninvasive apparatus and method for measuring mechanical performance of the heart using periodic or for continuous monitoring and recording parameters related to blood flow and pressure by peripherally deployed arterial sensors.

### BACKGROUND OF THE INVENTION

Heart muscle ischemia due to coronary artery diseases is one of the leading causes of death in the world; in the United States alone, it affects more than 13 million people. Myocardial ischemia can be defined as a decrease in the supply of blood to the heart, and more precisely as an imbalance between the supply and demand of myocardial oxygen. In most clinical situations, the reason for this imbalance is inadequate perfusion of the myocardium due to obstructions or stenosis of the coronary arteries. The ischemia can last a few seconds or persist for minutes or even hours, causing transient or permanent damage to the heart muscle. Each year, an estimated amount of 1 million Americans will have a new or recurrent coronary attack while more than 40% of the people experiencing coronary attack are expected to die resulting from it.

In order to monitor ischemic incidents and especially recurring ones, population at risk may connect to a cardiac center through a telephone line. Today, ambulatory monitoring of these patients or elderly population is performed using trans-telephonic electrocardiography (TTE). There are several disadvantages in using TTE:
1. TTE requires the patient to be symptomatic. However, 40-70% of transient ischemic episodes are silent, not associated with angina chest pain or any other symptoms.

2. TTE requires the patient to connect electrodes to his or her body, activate a recorder, and phone the cardiac center and trans-telephonic transmit the ECG. This is a complicated and an error-prone procedure, especially when performed by a patient suffering from these symptoms.

3. ECG test was shown in studies to have low sensitivity for diagnosis of ischemia (about 60%).

Experimental and clinical studies in the cardiologic literature and other references indicate that changes in the cardiac mechanical performance occur relatively early when an incidence of ischemia takes place, and indexes reflecting the mechanical performance of the heart are more sensitive than Electrocardiographs (ECG) changes or subjective symptoms for detecting myocardial ischemia. See: Kayden et al., “Validation of Continuous Radionucleide Left Ventricular Functioning Monitoring in Detecting Silent Myocardial Ischemia during Balloon Angioplasty of the Left Anterior Descending Coronary Artery”, Am. J. Cardiol. 67, 1339-1343 (1991), In this work the authors used balloon inflation in the course of transluminal coronary angioplasty as a human model of transient myocardial ischemia due to acute reduction of coronary blood flow, showing that 17/18 inflation were associated with a significant decrease in Left Ventricular Ejection Fraction, in contrast, there was chest pain in only 10 inflation's and ECG changes in 7.

In a work by T. Sharir et. al. in cooperation with one of the inventors of the present invention it was shown that the rate of pressure rise during the cardiac ejection phase increases with physical effort and decreases with infusion of vasodilating drugs. See: Sharir T, Marmor A, Ting C T, Chen J W, Liu C P, Chang M S, Yin F C P and Kass D A. Validation of a method for noninvasive measurement of central arterial pressure. Hypertension 1993, 21 74-82. The device used in that work is based in part on measurements of peripheral flow parameters and is described also in U.S. Pat. No. 5,199,438.

As explained above, it is desirable to provide a device that could monitor cardiac mechanical performance, especially among population at risk of cardiac malfunction. Monitoring should be performed either periodically or continuously, independently of clinical sympthomatology. As a consequence of the observations in the previous paragraph, such monitoring can be performed by measuring changes in parameters characterizing the flow of blood in the arm non-invasively and therefore providing an early detection of the cardiac pump impairment induced by ischemia. Noninvasive methods exist for measuring some of these parameters. An example of these methods is Doppler technique in which ultrasonic sound waves are transmitted through the skin roughly parallel to the blood flow direction, and variations in the ultrasonic frequency are sensed to determine the blood flow velocity. Alternative solutions for monitoring of the peripheral flow are based on various electromagnetic sensors. Examples of electromagnetic sensors were disclosed in U.S. Pat. No. 4,412,545 by Okino et al. “Electromagnetic Blood Flowmeter” and in PCT/IL01/00583 (Gorenberg et al.), titled APPARATUS AND METHOD FOR NON—INVASIVE MONITORING OF HEART PERFORMANCE, published as WO/02/00094.

However, the peripheral blood flow on its own is not a proven indicator of cardiac performance. Hence it is desired to measure peripheral hemodynamic parameters more indicative of the cardiac performance.

Additional sensors for non-invasive measurements of hemodynamic parameters have also been proposed. The patents U.S. Pat. No. 5,095,912 (Tomita), U.S. Pat. No. 5,301,675 (Tomita), U.S. Pat. No. 5,316,005 (Tomita), U.S. Pat. No. 5,388,585 (Tomita), U.S. Pat. No. 5,406,954 (Tomita), U.S. Pat. No. 5,423,324 (Tomita), U.S. Pat. No. 5,651,369 (Tomita) and U.S. Pat. No. 6,231,523 (Tomita), disclose devices for detecting pressure waves coupled with cuffs over the upper arm, resembling in some ways certain embodiments of the present invention. However, the inventions disclosed herein below are different both is essence and in details from Tomita in mechanical configuration, the specific blood pressure at which the measurement is performed, the algorithms for data processing and the purpose of the measurement.

U.S. Pat. No. 6,319,205 (Goor) and U.S. Pat. No. 6,322,515 (Goor) disclose an apparatus and method for monitoring physiological changes by performing a continuous monitoring of the arterial tone at the digit of the subject. Some of the embodiments in Goor involve mounting a cuff around the digit, application of pressure and monitoring the tone at the extreme end of the digit. However, also this invention is different in essence and in details from the present invention as explained hereinafter.

U.S. Pat. No. 5,503,156 (Millar) disclose a noninvasive pulse transducer for simultaneously measuring pulse pressure and velocity. The sensor disclosed in Millar's invention may have application in hemodynamic measurements but it is not specifically related to the present invention.

U.S. Pat. No. 5,199,438 (Pearlman) discusses a device for measuring cardiac power, incorporating components for measuring pressure waveform peripherally. The device and components in Pearlman are different both in essence and in details from the ones disclosed in this patent; moreover, they have a different purpose.

The present invention is also aimed at determining relative cardiac performance under stress. The techniques currently in use for detecting myocardial ischemia elaborated during exercise tests are summarized next:

1) ECG (Electrocardiography): As mentioned herein above, ECG has a relatively low sensitivity and specificity.

2) Stress echocardiography with Dobutamine infusion: This technique is based on performing two-dimensional ultrasonic imaging of the walls of the heart while infusing controlled doses of Dobutamine. Continuing improvements in this technique have increased the predictive diagnostic value of stress echo to approximately 75%-80. The test is labor intensive and professionally demanding requiring highly skilled personnel.

3) Nuclear imaging technologies. Radioactive isotopes are injected intravenously at peak physical effort or after the induction of pharmacological effort by Dobutamine infusion. A second intravenous dose of the same isotope is applied after the first dose is washed out and the patient is at rest. That procedure enables the physician to distinguish between filling defects due to infarcted regions versus transient filling defects in demand-related ischemic segments.

Presently, nuclear imaging methods appear to be the best available non-invasive procedures in clinical routines for ischemia detection for use after a positive result was obtained with ECG, or based on the physician's assessment of the patient. Reliability is in the range of 82-85%.

Patients deemed to have a significant degree of demand related myocardial ischemia on the basis of the diagnostic tests described herein above are usually further referred for cardiac catheterization and coronary angiography, which is the most invasive, but also the most definitive diagnostic test available.

The present invention is also aimed at aiding in ruling-out cardiac-related problems at a hospital's Emergency Room. Each year, about six million people are hospitalized at the U.S. alone after arriving at the Emergency Room complaining of chest pain. Most of these people are released one or more days after with a non-cardiac diagnostic. It has been found that the parameter computed using the present apparatus and method can be used to rule-out a substantial percentage of the false hospitalization, by verifying that the such parameter is larger than a fixed threshold, say 170.

### BRIEF DESCRIPTION OF THE INVENTION

It is an object of the present invention to provide a new and unique noninvasive device and method for monitoring, periodically or continuously, the heart mechanical performance. The main object is to measure the velocity at which the flow of blood propagates in a segment of a peripheral artery which is collapsed under external pressure. It is to be understood herein below, that flow of blood does not mean the velocity of the blood over a given cross section only, but rather the physical quantities characterizing the flow of blood: velocity, pressure and cross-section and the corresponding wave phenomena. It is also to be understood herein below that artery collapse means a substantial reduction of the cross-section of the artery and not necessarily that the artery be completely closed. In the medical art, this is sometimes referred as “partial” as opposed to “total” collapse. The inventors have found that the rate is highly correlated to the rate of pressure rise in the aorta during the ejection phase and is an indicator of ischemia state.

It is another object of the present invention to provide a new and unique device and method for monitoring the mechanical performance of the heart while the device is preferably mounted on the upper arm, the lower arm or the wrist, so that comfortable measurements conditions are met. The device may be mounted on another peripheral organ or area that meets the requirements of which blood flow may be measured without interference.

It is an additional object of the present invention to provide a new device that alerts patents to seek for immediate medical assistance when their heart performance is deteriorating.

It is yet another object of the present invention to provide a new device that facilitates true diagnosis in cases of ischemia so that false positive and false negatives ECG interpretation is avoided.

An additional object of the present invention is to provide a new device and method that facilitates evaluation of ischemia severity.

Yet, it is an additional object of the present invention to provide a new and unique device and method for recording and storing synchronized ECG signals with parameters that are correlated to the mechanical cardiac performance for relatively long periods of time (24-48 hours or even more) so as to provide an improved Holter system.

It is yet another object of the present invention to provide a new device to facilitate the diagnosis of obstructive sleep apnea syndrome by monitoring changes in peripheral vascular resistance (PVR).

It is yet another object of the present invention to provide a new device to facilitate the diagnosis of endothelial dysfunction, by monitoring changes in the flow of blood under mechanical or chemical extrinsic changes.

It is yet another object of the present invention to provide a new device and method to facilitate ruling out potential cardiac dysfunction, by comparing the parameter described herein below against a pre-fixed threshold.

There is thus provided, in accordance with a preferred embodiment of the present invention, a non-invasive apparatus for measuring cardiac mechanical performance of a patient, the apparatus comprising:
a pressure applying element mountable on a limb of the patient for applying pressure high enough to make a segment of an artery within the limb achieve a collapsed state and partially or totally empty it from blood at least momentarily;
at least one of a plurality of sensors coupled to said pressure applying element, sensing mechanical changes corresponding to volumetric changes in the artery as the artery progressively recuperates from its collapsed state;
a processing unit communicating with said at least one of a plurality of sensors for receiving output corresponding to the mechanical changes from said at least one of a plurality of sensors and computing factors correlated with blood flow and calculate parameters indicating heart performance.

Furthermore, in accordance with a preferred embodiment of the present invention, the pressure applying element is an inflatable cuff.

Furthermore, in accordance with a preferred embodiment of the present invention, the pressure applying element is an inflatable cuff, divided into a plurality of inflatable segments.

Furthermore, in accordance with a preferred embodiment of the present invention, the inflatable cuff is divided into at least two inflatable segments, and wherein said at least one of a plurality of sensors comprise at least two sensor transducers for detecting pressure changes within the segment, each transducer corresponding to a different segment.

Furthermore, in accordance with a preferred embodiment of the present invention, the pressure applying element is operated by a pneumatic system comprising a pump for increasing the pressure within the cuff, and valves for releasing the pressure from the cuff.

Furthermore, in accordance with a preferred embodiment of the present invention, the pressure applying element is driven by an electrical motor.

Furthermore, in accordance with a preferred embodiment of the present invention, the pressure applying element is coupled to a bracelet having a diameter which is automatically adjustable.

Furthermore, in accordance with a preferred embodiment of the present invention, the bracelet consists of a strap and wherein bracelet's diameter may be increased or decreased by turning a screw operated by a motor to which the strap is attached.

Furthermore, in accordance with a preferred embodiment of the present invention, the pressure applying element is hydraulically operated.

Furthermore, in accordance with a preferred embodiment of the present invention, the pressure applying element comprises said at least one of the plurality of cushions held against the limb by a rigid bridge.

Furthermore, in accordance with a preferred embodiment of the present invention, the cushions are inflatable.

Furthermore, in accordance with a preferred embodiment of the present invention, said at least one of the plurality of cushions consist of two such cushions, filled with ferromagnetic fluid that transforms from liquid to solid by application of magnetic flux, and electromagnetic coil provided adjacent each cushion, for inducing magnetic flux.

Furthermore, in accordance with a preferred embodiment of the present invention, the pressure applying element comprises at least one of a plurality of cushions held against the limb by a rigid bridge, and wherein said at least one of a plurality of sensors comprises deformation sensors, sensing deformation changes of said at least one of the plurality of cushions.

Furthermore, in accordance with a preferred embodiment of the present invention, said at least one of the plurality of cushions is inflatable.

Furthermore, in accordance with a preferred embodiment of the present invention, said at least one of the plurality of cushions is filled with hydraulic fluid.

Furthermore, in accordance with a preferred embodiment of the present invention, the deformation sensors comprise an array of capacitors wherein the mechanical changes are determined by measuring changes in the capacitance of the capacitors, due to deformation changes.

Furthermore, in accordance with a preferred embodiment of the present invention, the pressure applying element comprises at least one cushion held against the limb by at least one of a plurality of pivotal rigid bridges, provided with gyroscopic sensor to sense rotational velocity of said at least one of a plurality of pivotal rigid bridges.

Furthermore, in accordance with a preferred embodiment of the present invention, said at least one of a plurality of pivotal rigid bridges comprise two pivotal bridges.

Furthermore, in accordance with a preferred embodiment of the present invention, the two pivotal bridges are coupled to a third pivotal bridge.

Furthermore, in accordance with a preferred embodiment of the present invention, said at least one of a plurality of sensors include an array of deformation transducers.

Furthermore, in accordance with a preferred embodiment of the present invention, said at least one of a plurality of sensors include an array of piezoelectric transducers.

Furthermore, in accordance with a preferred embodiment of the present invention, said at least one of a plurality of sensors include an array of conducting rubber.

Furthermore, in accordance with a preferred embodiment of the present invention, the apparatus further comprises output means.

Furthermore, in accordance with a preferred embodiment of the present invention, the apparatus further comprises memory unit.

Furthermore, in accordance with a preferred embodiment of the present invention, the apparatus further comprises means to communicate with a computer, network or a telephone system.

Furthermore, in accordance with a preferred embodiment of the present invention, the pressure applying element is capable of applying pressure sufficient to cause a collapse of the artery just momentarily during a diastolic phase of the patient.

Furthermore, in accordance with a preferred embodiment of the present invention, a control system is used to maintain the applied pressure over a period of time substantially at the mean artery pressure or another chosen pressure and factors correlated with cardiac performance are measured continuously.

Furthermore, in accordance with a preferred embodiment of the present invention, the measurement data is used to calculate the velocity at which parameters of the flow of blood propagate through the artery while the artery progressively recuperates from its collapsed state.

Furthermore, in accordance with a preferred embodiment of the present invention, the velocity of the propagation of the flow of blood is calculated by a fit of a theoretical curve to the combined data of plurality of sensors, each detecting deformation changes within corresponding segment or segments of the inflatable cuff.

Furthermore, in accordance with a preferred embodiment of the present invention, the velocity of the propagation of the flow of blood is calculated from the time difference between data of plurality of sensors, each detecting deformation changes within corresponding segment of the inflatable cuff.

Furthermore, in accordance with a preferred embodiment of the present invention, the velocity of the propagation of the flow of blood is calculated by a fit of a theoretical curve to data indicating sensor segment triggering time versus said segment position.

Furthermore, in accordance with a preferred embodiment of the present invention, there is provided a method for non-invasive measuring of changes in cardiac mechanical performance of a patient; the method comprising:
providing a pressure applying element mountable on a limb of the patient for applying pressure enough to make a longitudinal segment of an artery within the limb achieve a collapsed state and empty it from blood at least momentarily;
providing sensor coupled to the pressure applying element, sensing mechanical changes corresponding to volumetric changes in the artery as the artery progressively recuperates from its collapsed state;
providing processing unit communicating with the sensor for receiving output corresponding to the mechanical changes from the sensor and computing factors correlated with blood flow and calculate parameters indicating heart performance;
applying pressure on a portion a limb of a patient through which artery passes enough to collapse the artery preventing at least momentarily the flow of blood through the collapsed artery;
sensing mechanical changes corresponding to volumetric changes in the artery as the artery progressively recuperates from its collapsed state;
calculating parameters indicating heart performance.

Furthermore, in accordance with a preferred embodiment of the present invention, the pressure applied on the portion of the limb of the patient is initially larger than needed to completely collapse the artery, and wherein it is gradually reduced, sensing the mechanical changes correlating to the volumetric changes while the pressure is reduced.

Furthermore, in accordance with a preferred embodiment of the present invention, the method further comprises measuring blood pressure of the patient.

Furthermore, in accordance with a preferred embodiment of the present invention, the method further comprises measuring heart pulse rate of the patient.

Furthermore, in accordance with a preferred embodiment of the present invention, the method steps are carried out continuously over a period of time, in order to diagnose heart performance disorders.

Furthermore, in accordance with a preferred embodiment of the present invention, the method further comprises transmitting data to an external apparatus.

Finally, in accordance with a preferred embodiment of the present invention, the method is incorporated with Holter procedure, in order to detect artifacts and enhance reliability.

Further features of the present invention are explained herein below.

### BRIEF DESCRIPTION OF THE DRAWINGS

FIG. 1 illustrates variations in the cross-section area of an artery when subjected to an inter-wall pressure.

FIG. 2 illustrates variations in the internal pressure of a cuff, as the static pressure of the cuff is reduced. The peaks are caused by the artery when recovering from the collapsed state.

FIG. 3 illustrates a preferred embodiment of the present invention, with a cuff divided into two segments, a data acquisition, data processing unit and display, worn in upper arm.

FIG. 4 illustrates an alternative preferred embodiment of the present invention, with a display and a pressure applying band.

FIG. 5 illustrates a schematic diagram of the pneumatic components of a monitoring device in accordance with a preferred embodiment of the present invention.

FIG. 6 illustrates a schematic diagram of the electronic components of a monitoring device in accordance with a preferred embodiment of the present invention.

FIGS. 7a-e illustrate cross-sectional views of the progress of blood through a collapsed artery, and the induced mechanical changes within the cuff on the first preferred embodiment. The pressure changes within the cuff are shown in a chart below each drawing.

FIG. 8 illustrates a graph of the variations in internal pressure within a segment of the cuff following the opening of the collapsed artery. The graph is obtained after subtracting the static pressure that reduces monotonically, hence the O-baseline.

FIG. 9 illustrates a graph of the variations in internal pressure within a segment of the cuff, corresponding to the FIG. 8, with the minimum and maximum local values normalized between 0 and 1.

FIG. 10 illustrates the normalized pressure reading output of the two cuff segments of the first preferred embodiment. The middle point, corresponding to an abscissa of 0.5 is important since the measurement is linear there and the middle point of the second cuff segment should correspond to a value of 1.5 of the first segment.

FIG. 11 illustrates the location for positioning the monitoring device over a wrist, in accordance with a preferred embodiment of the present invention.

FIGS. 12a-c illustrate the pressure applied on an artery in the wrist and the progress of blood through the artery. Pressure changes due to the artery recovering from collapse are sensed by two cushions 1202-1203 which translate the variations into a rotation of a sensing bridge 1201 FIG. 13 illustrates an alternative embodiment to the one illustrated in FIG. 12, where the sensor is an inflatable cushion with sensing elements in the form of varying capacitors 1310.

FIG. 14 shows an electrical equivalent of the embodiment illustrated in FIG. 13.

FIG. 15 illustrates de behavior of the preferred embodiment of FIG. 13. The changes in cross section due to the artery recovering from collapsed reduce the distance to the capacitors plates 1510. These changes are measured by measuring the voltage drop across two opposite plates.

FIG. 16 shows a modification of the previous electrical equivalent FIG. 17 illustrates an alternative embodiment to the one illustrated in FIG. 12, where the rotational velocity of the plate attached to the inflatable cushions is measured by using a gyroscope.

FIG. 18 illustrates still another alternative embodiment to the one illustrated in FIG. 12, where two mechanical bridges are coupled by a rigid arc 1801. This configuration amplifies the mechanical movement which is sensed by two gyroscopes.

FIG. 19 illustrates a variation to 18, which is more involved from a mechanical point but achieves a better mechanical gain due to larger bridges.

FIG. 20 illustrates another variation to 18, which is more involved from a mechanical point but achieves a better mechanical gain due to larger bridges.

FIG. 21 illustrates a schematic representation of a sensor with a single cuff.

FIG. 22 illustrates how the pneumatic circuit is simplified when using a single cuff as in FIG. 21.

FIG. 23 illustrates how the electronic circuit is simplified when using a single cuff as in FIG. 21.

FIG. 24 illustrates the timing of the pulses sensed by the sensors embedded on the single cuff embodiment.

FIG. 25 illustrates the peak-by-peak computations performed by the algorithm.

FIG. 26 illustrates the variations in pulse-by-pulse measured velocity as a function of the quasi-static pressure illustrates the

FIG. 27 illustrates an alternative embodiment, where the sensor is position dependent and hence allows the measurement of the volume changes due to the artery recovering from collapse.

FIG. 28 illustrates a particular implementation of the embodiment where the sensor is a flexible conductive film strain gauge 2810.

FIG. 29 illustrates an alternative embodiment for the sensor in FIG. 27, where the sensor is a piezoelectric film with varying resistance.

### DETAILED DESCRIPTION OF THE INVENTION AND DRAWINGS

The present invention provides a noninvasive device and method for peripheral monitoring of the mechanical performance of the heart muscle in a periodic manner, continuously or per user request. The noninvasive monitoring device is relatively small in dimensions, therefore portable and may be designed as a cuff or bracelet that may be worn on the upper arm, lower arm, or wrist of a patient, acquire information and store it or transmit it to a processing or display device.

The inventors of the present invention found and demonstrated a clear and distinct correlation between indexes of heart performance measured centrally and peripherally, therefore, the convenience of such a device is apparent and appealing. See PCT/IL01/00583 (Gorenberg et al.), APPARATUS AND METHOD FOR NON—INVASIVE MONITORING OF HEART PERFORMANCE, published as WO/02/00094.

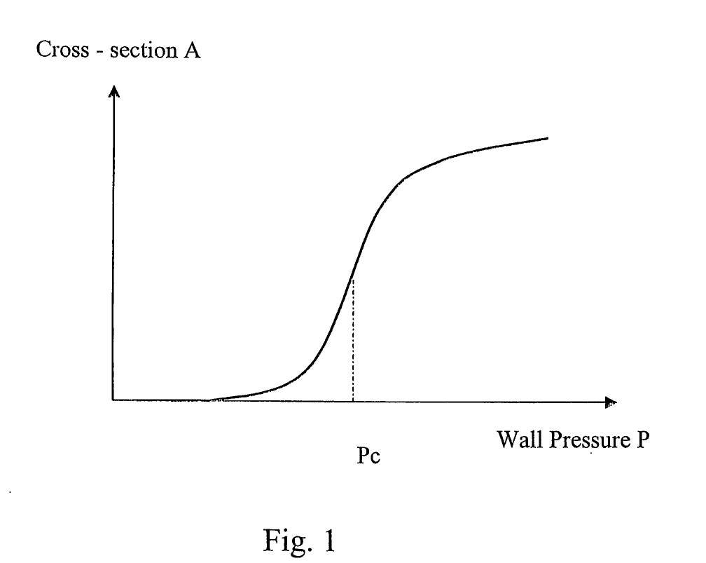

The invention disclosed herein uses the collapsible nature of arteries to empty, either partially or completely, the vessel from blood in between diastole. A sufficiently large negative pressure on an artery wall makes it collapse, emptying the vessel, either partially or completely, from blood. As the external pressure decreases, the artery recovers its original shape and hence allows the free circulation of blood. FIG. 1, schematically shows the variation of an artery cross section as a function of intramural pressure.

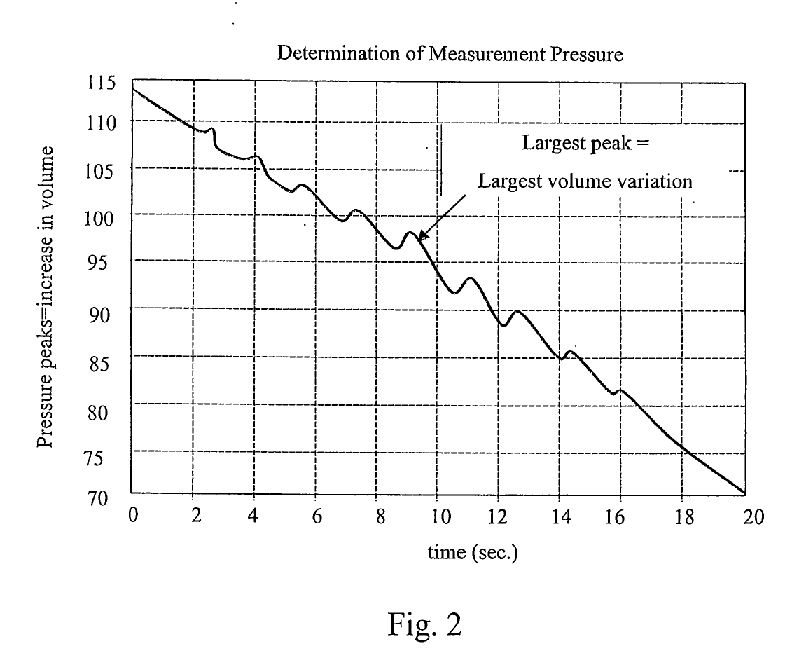

Now suppose that a sufficiently large external pressure is applied so that the artery collapses. This external pressure can be achieved by means of an external device applying pressure on the organ where the artery is located, preferably the arm or wrist, but also in any other peripheral part of the body (preferably, but not limited to a limb). When the internal pressure peak driving the blood flow reaches the section where the external pressure is applied, it opens the artery, thus allowing the flow of blood. The recuperation of the artery from collapsed state to normal state does not occur instantaneously. We consider a situation where the artery is collapsed during the diastolic phase of the cardiac cycle due to external pressure by a cuff. Substantially there is no flow through the artery, the blood forms a standing column of liquid from the aorta to the proximal side of the cuff and the pressure at the proximal side of the cuff is substantially the same as in the aorta. As the blood pressure builds up in the aorta during the ejection phase, a pressure wave is transmitted through the collapsed artery, from the proximal to distal side, recuperation the blood vessel and allowing blood flow. The behavior of the internal pressure as a function of the quasi-static pressure is illustrated in FIG. 2. The velocity of the pressure wave is substantially lower than the velocity of pressure wave in large arteries that are not disturbed by an external pressure. Indeed, under total or partial collapse the velocity of propagation of pressure within the artery and the velocity of propagation of the blood flow approach one another.

Insofar, the velocity of the propagation of the flow of blood in a collapsed artery has not been used in medical devices and was not considered to have a diagnostic value. The inventors of the present invention have found that the velocity in the collapsed artery, denoted inhere below as Vₚ, depends strongly on the external cuff pressure relative to subject diastolic and systolic blood pressures, the rate of pressure rise in the aorta during the ejection phase and the compliance curve of the artery. Therefore, monitoring of this velocity is useful to monitor changes in cardiac mechanical performance affecting the rate of pressure rise in the aorta. Further, it is useful to diagnose the state of the arterial system and monitor effects of drugs, affecting the compliance curve.

Following the discussion above preferred embodiments of the apparatus for non-invasive monitoring of heart performance in accordance with the invention include the following main components:

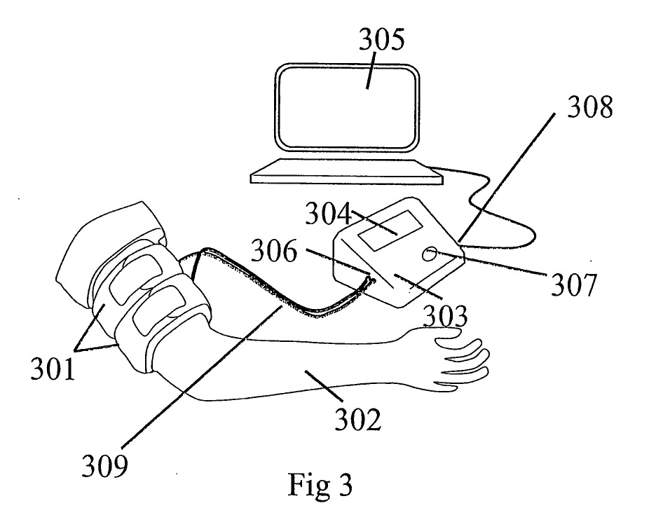

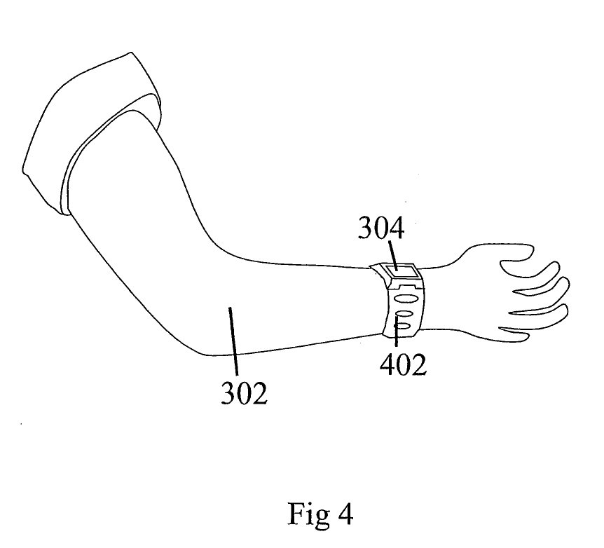

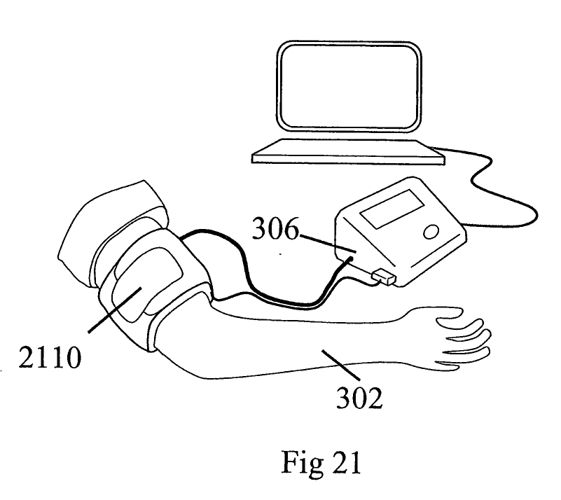

1. A device such as the cuff 2110 in FIG. 21, the double-cuff 301 in FIG. 3 and the mechanical band 402 in FIG. 4 for producing a collapse of an artery on the arm or wrist, based on applying external pressure. The exerted pressure must be sufficiently high to produce the collapse but low enough to allow recovery from collapse during the peak systolic phase.

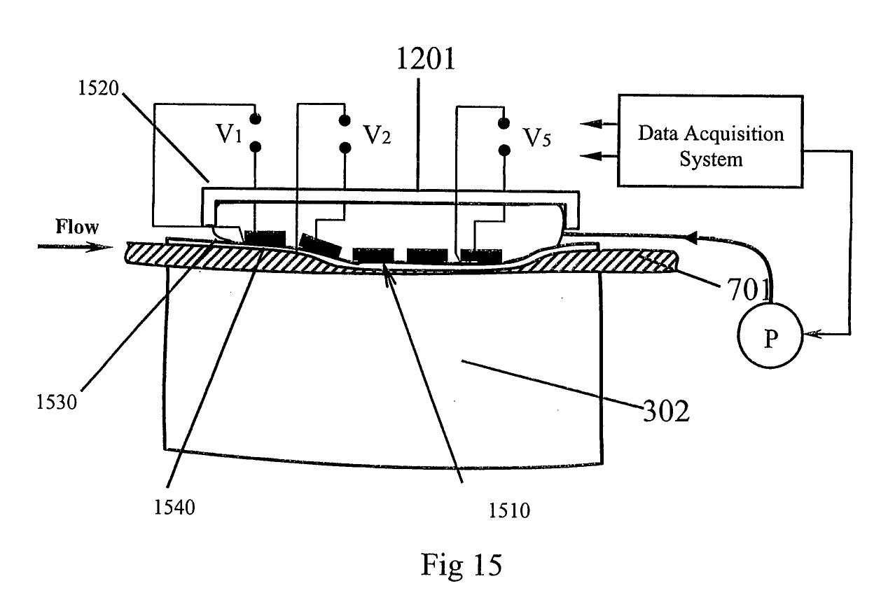

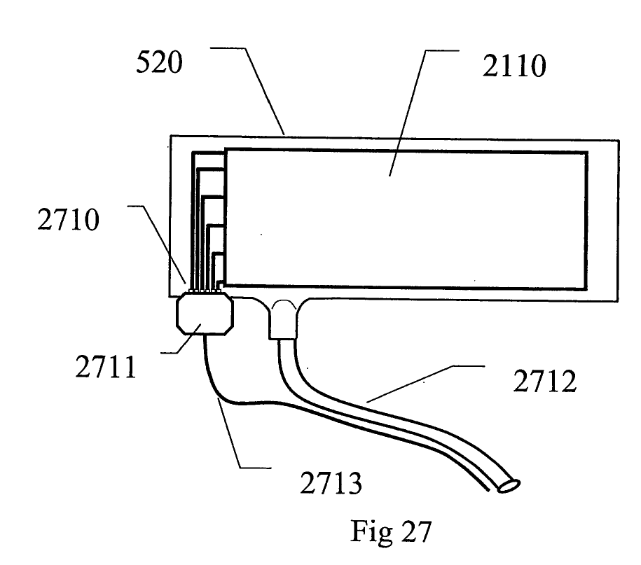

2. A sensor such as the double cuff 301 in FIG. 3, the capacitor sensor 1310 in FIG. 13, the deformation sensor 1510 in FIG. 15, the gyroscope 1701 in FIG. 17 to 21 and the generic deformation sensor 2110 in FIG. 27, for measuring the propagation of the flow of blood occurring when the blood flow opens the artery.

3. A processing unit such as 303 in FIG. 3 for recording, analyzing and preferably also storing the data retrieved from the sensor.

In one of the novel aspects of the invention disclosed hereinafter, the sensing unit uses a plurality of relatively short and simple sensors, and a numerical algorithm is used to combine the information from the plurality of sensors in a way that drastically increases the reliability, sensitivity and signal to noise ratio of the device. In alternative configurations, a single transversal deformation sensor may be used for providing a linear and reliable measurement of the progress of the opening of the artery (for example, FIG. 27b). Deformation may be measured in many ways, some of which include using piezoelectric transducers, optical sensors (with lasers, optical fibers etc.), conducting rubber, capacitors, and other types of sensors reacting to deformation. Three of these options are illustrated in FIG. 27 to 29.

The external pressure to be used during the measurement should be enough to collapse the artery in the diastolic phase so an increase in the internal blood pressure is sufficient to recover the artery from collapse to open. In preferred embodiments of the present invention this pressure is not determined a priori but rather obtained via an indirect measurement, which can be described as follows. First, a relatively large external pressure is applied to assure that the artery collapses in the region of the external pressure. Subsequently, the pressure is reduced gradually while monitoring changes in the cuff volume. During the reduction, at first no change in volume is observed since the internal blood pressure is not sufficient to open the artery at any phase of the heart cycle, but then at some point the artery opens and closes periodically following the pressure variations. As a result, one observes a graph as shown in FIG. 2.

The pulses in the pressure curve in FIG. 2 are proportional to the change in volume following the variations in the cross section of the artery resulting from the propagation of the flow of blood. The largest pulse amplitude represents the point at which the external pressure is large enough to collapse the artery, but a small increase in the internal blood pressure immediately causes a large change in the cross section of the vessel. Consequently, the pressure for taking the measurement can be roughly determined by applying a monotonically decreasing external pressure and then computing by means of a numerical algorithm the pressure at which the largest variation occurs. More detailed algorithms for the a-priory determination of the measurement point are disclosed herein below.

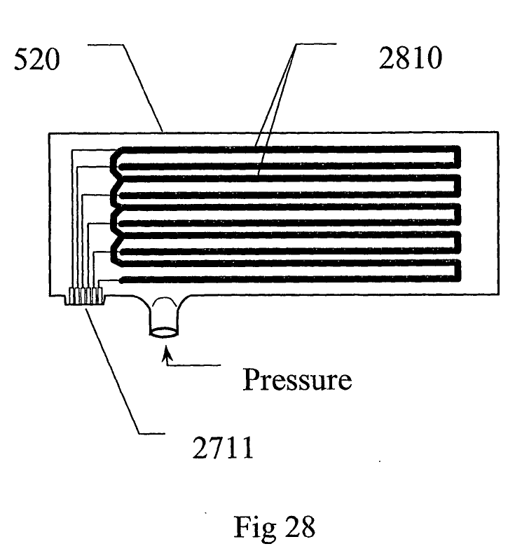

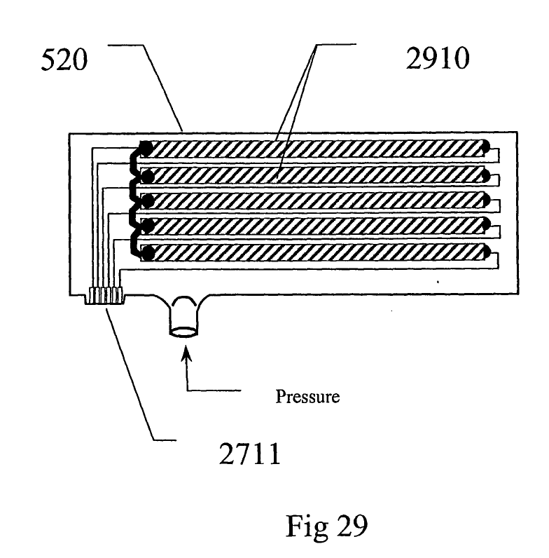

In a preferred embodiment the sensor is composed of 5 elements shown as 1510 in FIG. 15. The elements are positioned parallel to each other at distances of 18 mm apart. For the purpose of describing the apparatus, it is assumed herein below that the preferred sensor is made of a collection of piezoelectric elements. In a variation of this preferred embodiment, the sensor is a flexible conductive film strain gauge 2810 in FIG. 28, divided into independent sections that replace the sensor 1510 in FIG. 15. In another variation of this preferred embodiment, the sensor is a piezoelectric film with varying resistance, illustrated as 2910 in FIG. 29. It is understand that a person known in the art can modify the apparatus by using the technology of conducting rubber for measuring deformation, with minor modifications to the preferred embodiments illustrated in FIG. 15. The mechanical layout of the sensor is shown in a cross section FIG. 15. The sensor is constructed of several encasing layers: Adhesive tape 1530 such as Nashua type No. 398, aluminum foil 1520, patches of insulating tape 1540 and inner most piezoelectric elements 1510. The purpose of this design is to provide isolation from electrical noise and sufficient in-plane inflexibility so as to keep the distance between elements 1510 constant. On the other hand, the design allows transmission of the pressure variations from the arm 302 to sensors 1510 and avoids mechanical cross talk between the elements 1510. It is understood that other mechanical arrangements and distances between elements work as well.

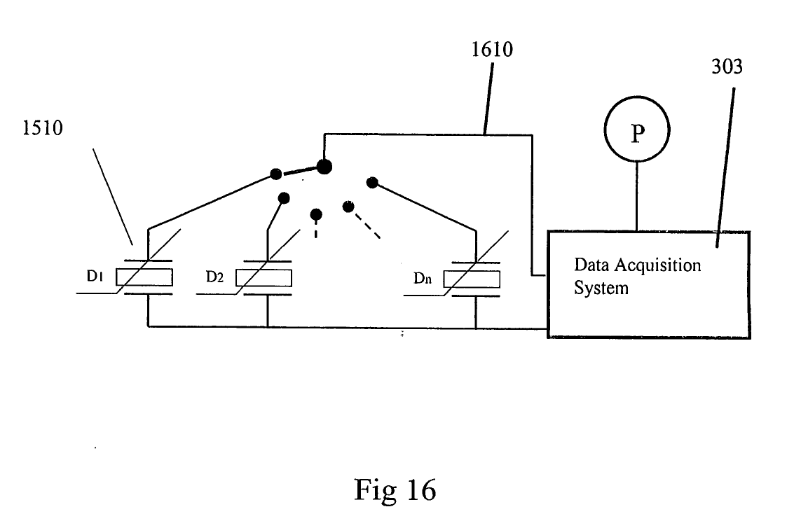

The electrical connection of elements 1510 is shown schematically in FIG. 16. The sensors 1510 are modeled by varying voltage sources 1610 such that each one has a pole connected to output signal V1-V5, respectively. The other pole of each element is connected to a common line. All lines are fed through connection line to the processing unit 303. In this embodiment, signals V1-V5 are transmitted through a shielded cable 1610. The shield is connected to the aluminum foils 1520 and to the case of monitor 303. In alternative embodiments, two separate output lines are connected to each element and transmit differential output to control box 303.

In a preferred embodiment a cuff with a width of 15 cm is used for upper arm mounting. The sensor is placed in the cuff such that the most proximal element is positioned 3.4 cm from the proximal edge of the cuff and the most distal element is placed at a distance of 3.4 cm from the distal edge of the cuff. All distance are measured to the longitudinal center of the sensors 1510. When mounted on the subject arm, the sensor has to be placed generally in the inner side of the arm generally parallel to the brachial artery. This can be achieved by proper marking of the desired positioning on the outside of the cuff. In order to reduce the sensitivity of the device to positioning relative to the brachial artery, it is possible to couple to each of elements 1510 a relatively rigid strip extending circumferentially around the arm and transmitting the pressure variations from the arm to the elements. However, the inventors have obtained consistent results also without using such strips.

In a preferred embodiment illustrated in FIG. 13, the sensors in FIG. 15 are replaced with capacitors with varying capacitance 1310. Placing a plate of conductive material 1310 and using the conducting material 1201 as a second plate implement these capacitors. It is well known in the art that the capacitance of these devices depend on the distance X1 to Xn between the plates.

In an alternative preferred embodiment shown in FIG. 27 the sensor is directly embedded on the cuff by using the technology of flexible conductive film strain gauge and the technology of piezoelectric film with varying resistance. In an alternative preferred embodiment the sensor is directly embedded on the cuff by using the technology of conducting rubber. In this embodiment, a collection of thin strips of the elastic material of the cuff are modified by injecting a conducting material that substantially reduces the resistance of the strips to electricity. Alternatively, the conductive rubber is embedded into a patch of material pasted on the cuff. The resistance of the strips depends on the elongation of the strips: when the strips are elongated in response to changes in the cross section of the arm, their resistance increases. An electric circuit, such as a Wheatstone Bridge is used to transduce the resistance into an electric potential, which is sampled by the same sampling circuit mentioned for the preferred embodiment discussed herein above.

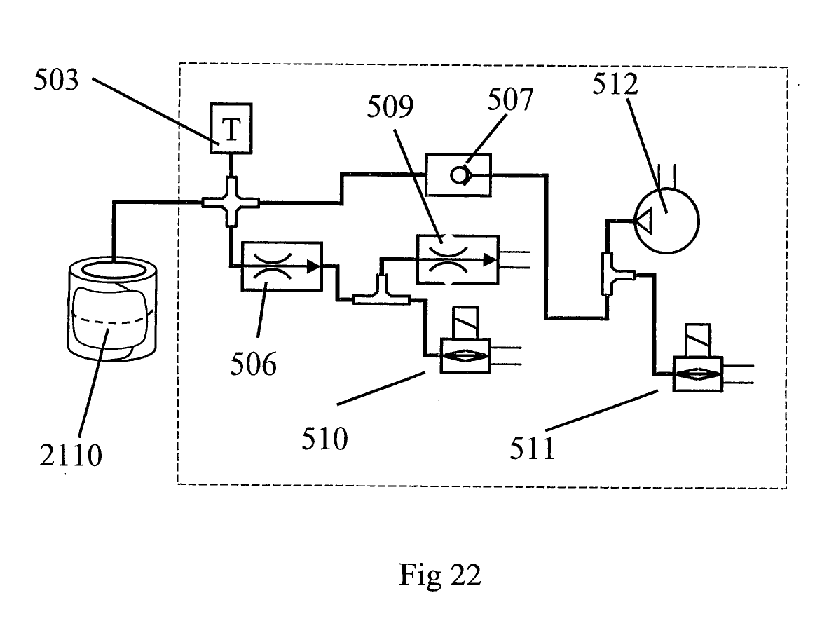

Reference is made to FIG. 15. The operation of the preferred embodiment of the present invention is described herein below with further details, using the piezo-resistive elements for illustration. It is clear that the same explanation applies to the capacitor sensor in FIG. 13, and the generic deformation sensor in FIG. 27 with its two preferred embodiment illustrated in FIGS. 28 and 29. The inflatable cuff 2110 is communicating with a sensor schematically represented by 306 for measuring the instantaneous pressure within the segment. In the preferred embodiment, typically this sensor is a micro machined membrane piezo-resistive transducer, such as the sensor manufactured by Motorola™ and marketed under the brand name of MTX2201, although other transducers of air pressure could also be used. A full cycle of the pneumatic components would look like this: referring to FIG. 22, with valves 510 and 511 closed, the air pump 512 pumps air to the inflatable cuff 2110. When the desired high pressure is reached, the pump stops working. The high pressure is typically above the patience's systolic pressure. Then, valve 510 is opened, so that the pressure to the right (respectful of the drawing) of the non-return, one-way, valves 507 drops and prevents the air from flowing back. The air flows from the cuff through the pressure regulators 506, and then through the regulator 509 such that the pressure in the cuff is reducing at typically 2 to 3 mmHg per second. While the air is flowing from the cuff, the pressure transducer 503 measures the internal pressure in the cuff. When the internal pressure of the cuff drops well below the diastolic pressure, measurements are stopped. Then, valve 511 is opened to allow the remaining air to exit the cuff. At this point a new cycle may begin.

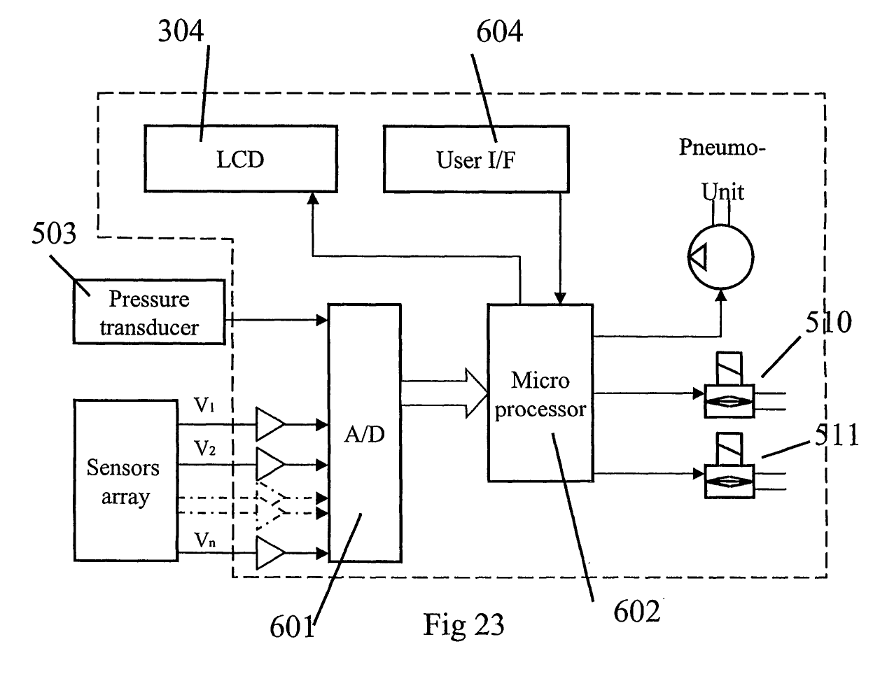

The electronic circuit of a preferred embodiment of the present invention is illustrated in FIG. 23. The analog measurement of the pressure sensors 503 is amplified by operational amplifier such as AD620 from Analog Devices and digitized by an A/D converter (jointly shown as 601 in the figure). Piezoelectric elements 1510 outputs V1-V5 are fed into operational amplifiers such as OP07 from Analog Devices and also digitized by A/D converter (also shown as 601 in the figure). The resulting data is processed by micro-processor 602 using the algorithm described herein below. The results of the calculations are shown to the user on display 304 (for example LCD). In an alternative embodiment, calculations (or measurements, or graphic data) are displayed on a monitor 305 in FIG. 3. The user may input required data or commands using interface (such as keyboard) 604. The micro-processor 602 also controls the pneumatic circuit, by operating the air pump 512 and closing and opening the solenoid valves 510, 511. Measurement results may be stored in a memory unit. In alternative embodiments of the present invention, non-linear amplifiers are used to condition the piezoelectric signals V1-V5 so as to improve signal to noise ratio while increasing the dynamic range. In further preferred embodiments, the pressure transducer signal may be split—one branch going through a low pass filter to yield the un-modulated quasi static pressure signal while the other branch goes through a high pass filter to yield a DC subtracted modulations function.

Referring now to FIG. 4, in preferred embodiments of the present invention the monitoring device and the analyzer are incorporated in a device worn by the patient on the wrist in the shape of a bracelet 402, secured to the wrist by a strap. The bracelet contains an inflatable or mechanical device for applying a gradually reducing pressure and a sensor comprising piezoelectric elements, as in the case of the previous embodiment. Housing 304 houses the electronic and mechanical components of the device, as explained hereinafter.

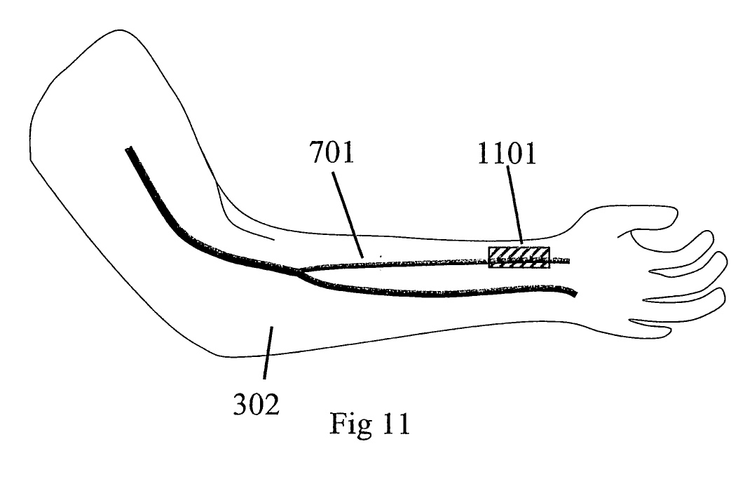

Comparing the embodiments disclosed in FIG. 3, 21 and FIG. 4, it is noted that while the artery in the upper arm passes deep inside the arm, in the wrist the artery is located close to the surface of the limb, which is reflected in the respective mechanical embodiments. In the upper arm pressure is applied in circumferential aspect, using a cuff surrounding the arm circumferentially as shown in FIG. 3. For the wrist apparatus shown in FIG. 4 it is sufficient to focus the pressure on a small area adjacent to the radial artery as illustrated in FIG. 11, although it is also possible to use a device applying circumferential pressure. Both embodiments include an inflatable or mechanical mechanism for applying sufficient pressure to assure a complete collapse of the respective artery, and subsequently decreasing the pressure, to allow for a gradual and progressive opening of same artery.

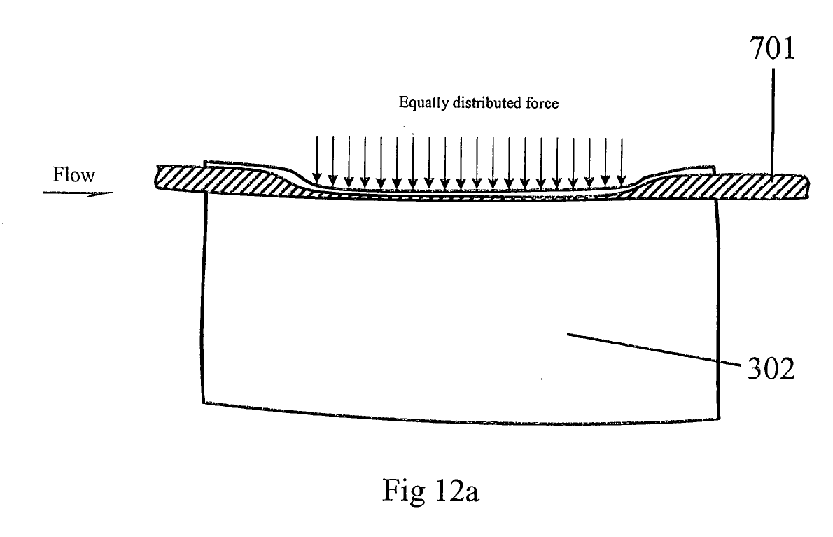

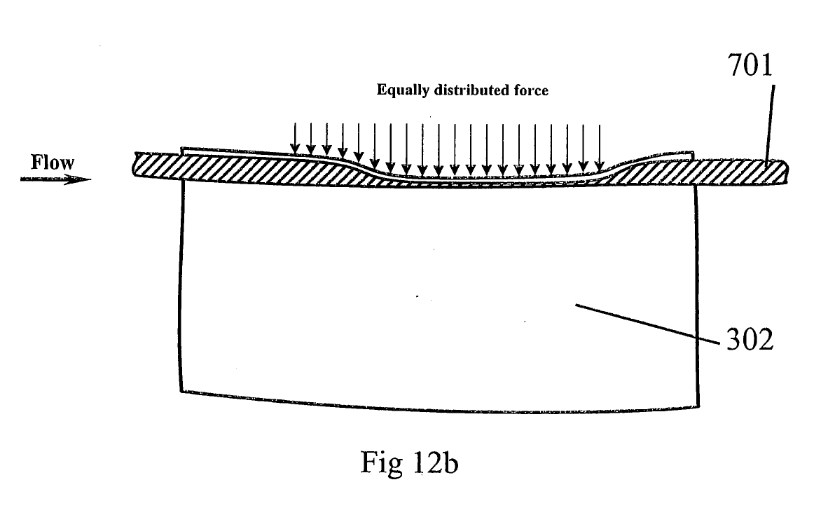

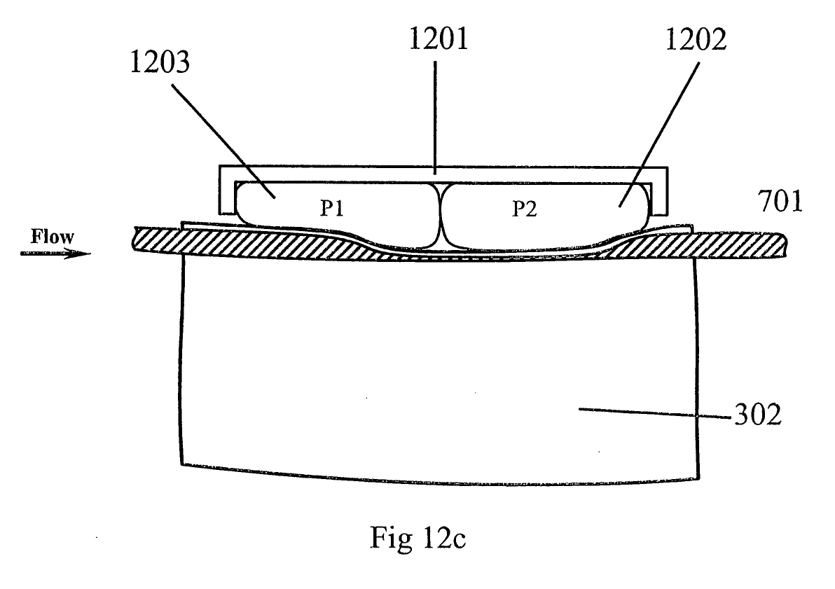

The operation of the preferred is as follows: External pressure is applied to a level above the systolic blood pressure, thereby occluding the artery and stopping blood flow as shown in FIG. 12a. Subsequently the pressure is decreased at a rate of typically 2-3 mmHg per second while the pressure and piezoelectric transducers data is acquired. When the pressure is below diastolic value, the measurement stops and the remaining pressure is released.

While the external pressure is decreased from above systolic to below diastolic blood pressure, modulations are observed in the pressure readout due to the periodic collapse and recovery of the artery, as shown in FIG. 2. Modulation function is then deduced as described inhere below and shown in FIG. 12. As the external pressure decreases, the amplitude of the modulations increases until the external pressure is approximately at the mean artery pressure and then it decreases.

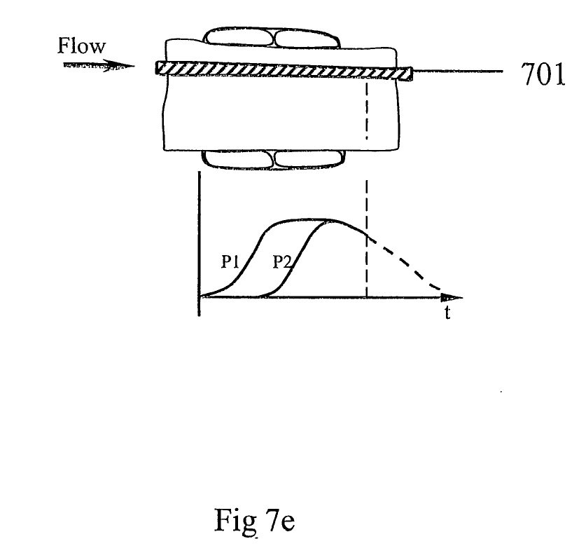

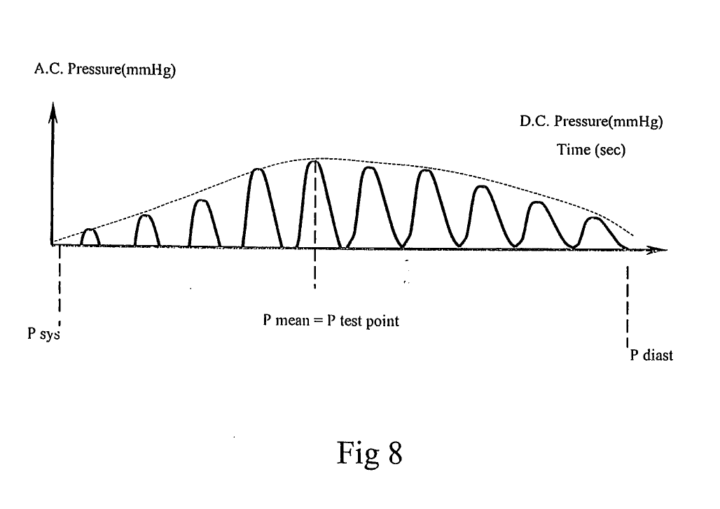

Calculation of the modulation function is carried out by subtracting from the pressure data the quasi static pressure. This can be done, for example by approximating the quasi-static pressure as a function of time using a low order polynomial fit to the pressure data, or by smoothing of the measurements data using appropriate low pass filter. The subtraction of the quasi-static pressure results in a time data containing instantaneous variations in internal pressure following the opening of the arteries. FIG. 8 shows a typical example of the resulting data. Alternative solution is to extract the modulations data from the pressure data by an electronic filter prior to digitization of the pressure signal.

The initial pumping pressure may be predetermined. However, in a preferred embodiment of the present invention, the modulation function is deduced while the measurement still takes place. In the early stage of deflation, if the amplitude is not sufficiently increasing from heart beat to heart beat, it is assumed the initial pressure is not sufficiently high, the pump is automatically operated to increase the initial pressure higher and the acquisition restarts. If, on the other hand, the modulation function increases and than sufficiently reduced, it is assumed the pressure is already below diastolic pressure and the measurement automatically stops.

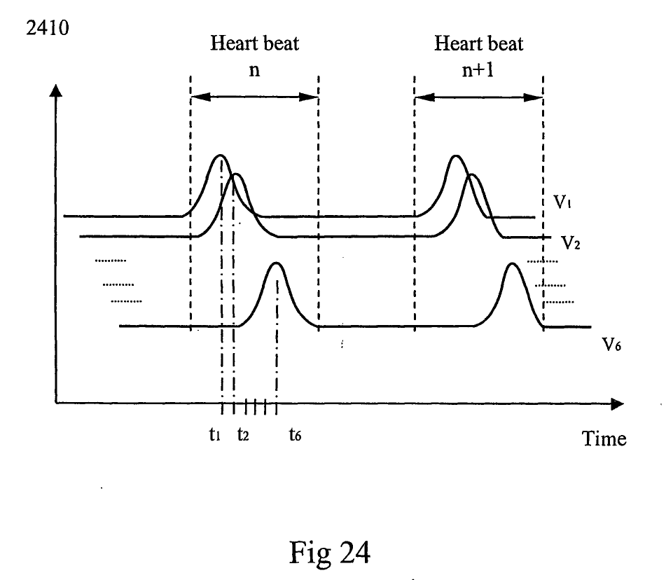

FIG. 24 is a schematic presentation of the data obtained from the array of piezoelectric transducers 1510. For each of the piezoelectric elements there is a sequence of pulses corresponding to heartbeats. Further, the pulses from the different elements in the array form groups corresponding to heartbeats. The groups are ordered in time from the most proximal element to most distal element. In FIG. 24 two heartbeats are shown with the corresponding groups of pulses.

The pulses are analyzed as follows:

1. Pulses are detected by a peak detection algorithm as known in the art. Peaks of amplitude below a pre-determined value are excluded. In a preferred embodiment using linear amplifiers a threshold of 10% from the total range is use. Other criteria are that there are at least a given number of pulses within a given time window. In a preferred embodiment using array of 6 piezoelectric elements, at least 4 pulses are required to be within a time window of 0.2 second. In some embodiments it is also required that the pulses will coincide with the systolic phase determined from the pressure modulations signal. Only pulses satisfying the above criteria are used for further analysis.

2. For each of the piezoelectric element pulses, the timing is determined. In a preferred embodiment, the timing is taken at the time of the pulse peak. In other preferred embodiments it is the time of maximum positive slope of the signal. Other ways to define the time is the crossing point of the leading edge of the pulse and a pre-set threshold or to determine the time at which the leading edge reaches a pre-set fraction of the pulse amplitude.

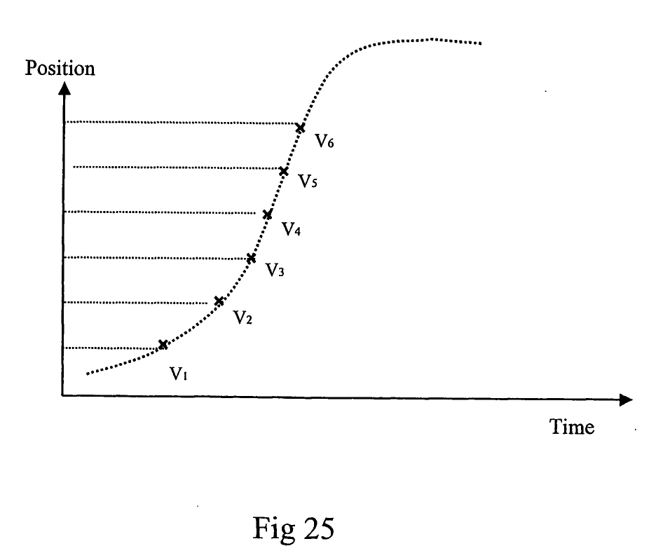

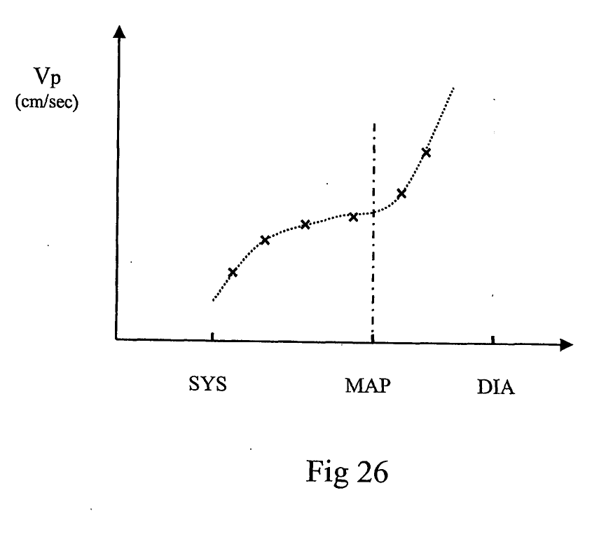

3. For each heart beat in the range between systolic and diastolic blood pressures, the pulses timing, as measured relative to the most proximal element timing is plotted versus the position, as shown in FIG. 25. The data is fitted to a simple curve to yield a value of the average pulse wave velocity during that heart pulse. In the preferred embodiment, this simple curve is a straight line. A fit to a straight line provides sufficiently accurate results although fits to other functions can be used as well. Typical results are plotted in FIG. 26, showing the pulse wave velocity V_{P} for several heart beats between the systolic SYS and diastolic DIA blood pressures. MAP denotes the mean artery pressure.

4. For clinical diagnostics purpose, it is useful to refer to the pulse velocity at the MAP. The value of V_{P} at the MAP is determined by a fit of the curve in FIG. 26 to a straight line or to a low order polynomial, over the entire range or for several pulses above and below the MAP. The values of the MAP is determined as the value at which the pressure modulation amplitude is at maximum or according to the formula: MAP=DIA+(SYS−DIA)/3.

However, for other diagnostic purposes other parameters can be deduced from the data in FIG. 26, such as the values of V_{P} at SYS or DIA, the ratio between the two or the slope of curve in FIG. 30c.

The blood pressures SYS and DIA are determined by one of three methods or a combination thereof:

1. A prior independent measurement by any of the devices available in the industry.

2. From the pressure modulation function (FIG. 8) by any of the algorithms known in the art.

3. Directly from the piezoelectric elements data. The inventors have found that piezoelectric pulses satisfying the criteria set forth above appear consistently in the range between DIA and SYS and usually do not appear in pressures above systolic or below diastolic. Hence, the subject diastolic blood pressure can be determined to an acceptable accuracy as the lowest cuff pressure at which there is complying piezoelectric data minus 0.5 of the pressure difference between heart beats. The subject systolic blood pressure is the highest pressure at which there is complying piezoelectric data plus 0.5 of the pressure difference between heart beats.

Further, the subject heart pulse rate and stability of pulse rate can be deduced from the measured data by measuring the average and beat-to-beat time difference between heart pulses, as appears in the pressure modulations data or the piezoelectric pulse data.

Reference is now made to FIG. 3 illustrating an alternative noninvasive devices for monitoring heart mechanical performance in accordance with a preferred embodiment of the present invention.

In a preferred embodiment of the present invention, a monitoring device is worn by a patient on the arm (FIG. 3) in the shape of a cuff 301. The cuff is designed to facilitate the collapse of a blood vessel (typically an artery) within the arm and measure the incremental opening of the artery following the progress of blood through the vessel.

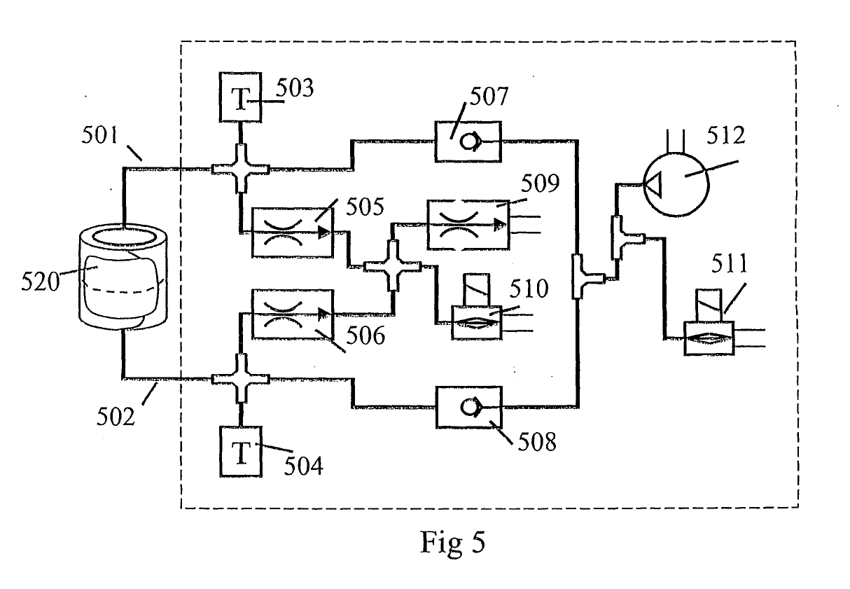

In the particular embodiment of FIG. 3 the cuff 301 comprises two adjacent inflatable segments that upon inflation exert, each, a pressure on the arm enough to collapse a blood vessel located under the cuff. However, in other similar embodiments more than two segments can be used. The cuff may be secured over the arm by fastening bands (similarly to blood pressure measuring cuffs). Each inflatable segment coupled to a pressure sensor (not shown in FIG. 3 but schematically represented in FIG. 5, as 503 and 504) that is separately connected to the control unit 303, a housed analyzer having readout display 304, optional user interface 307 and optional output socket 308 allowing the device to be connected to an external computer 305. The connection lines 309 may be pipes (if the pressure sensors are located in the reader—in order to transfer the pressure experienced within each inflatable segment to the sensors) or electric conductors (if the pressure sensors are positioned within the cuff and output electric signals). Control unit 304 includes electronic or pneumatic components as is described herein after. Note that throughout the present specification and claims by “sensor” is meant not only a single sensor but also a number of sensors or sensing means.

Referring back to FIG. 3 and FIG. 5, the operation of the arm-mounted embodiment of the present invention is described herein below with further details. The inflatable cuff 301 is divided into two independent inflatable segments. These segments have roughly the same size, and are located at a fixed predetermined distance between them. Each segment is communicating with a sensor (306) for measuring the instantaneous pressure within the segment. In a preferred embodiment, typically this sensor is a micro machined membrane piezo-resistive transducer, such as the sensor manufactured by Motorola™ and marketed under the brand name of MTX2201, although other transducers of air pressure could also be used.

Pneumatic arrangement is provided which keeps substantially the same static pressure in the two segments throughout the measurement. The pneumatic arrangement of this embodiment is illustrated in FIG. 5.

A full cycle of the pneumatic components would look like this: With valves 510 and 511 closed, the air pump 512 pumps air to the inflatable cuff segments 104, 106. When the desired high pressure is reached, the pump stops working. The high pressure is typically above the patience's systolic pressure. Then, valve 510 is opened, so that the pressure to the right (respectful of the drawing) of the non-return, one-way, valves 507 drops and prevents the air from flowing back. The air flows from each segment of the cuff through the pressure regulators 505, 506, and then through the regulator 510. While the air is flowing from the cuff, the pressure transducers 503 and 504 measure the internal pressure in each section of the cuff. When the internal pressure of the cuff drops well below the diastolic pressure, measurements are stopped. Then, valve 511 is opened to allow the remaining air to exit the cuff. At this point a new cycle may begin.

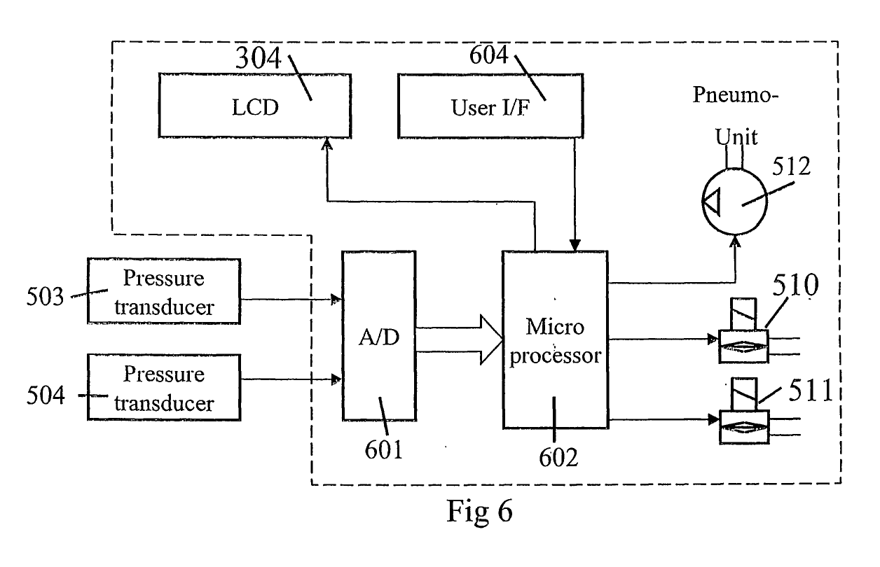

The electronic circuit of a preferred embodiment of the present invention is illustrated in FIG. 6. The analog measurements of the pressure sensors are preferably digitized using an A/D converter 601. The resulting data is processed by microprocessor 602 using the algorithm described herein below. The results of the calculations are shown to the user on display 304 (for example LCD). The user may input required data or commands using interface (such as keyboard) 118. The micro-processor 602 also controls the pneumatic circuit, by operating the air pump 512 and closing and opening the solenoid valves 510, 511. Measurement results may be stored in a memory unit.

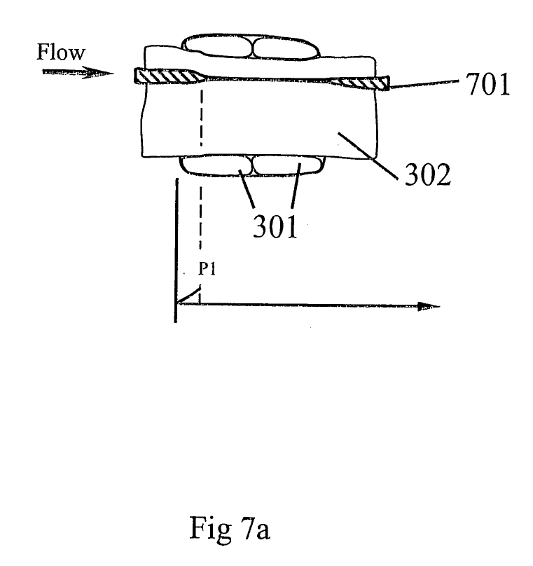

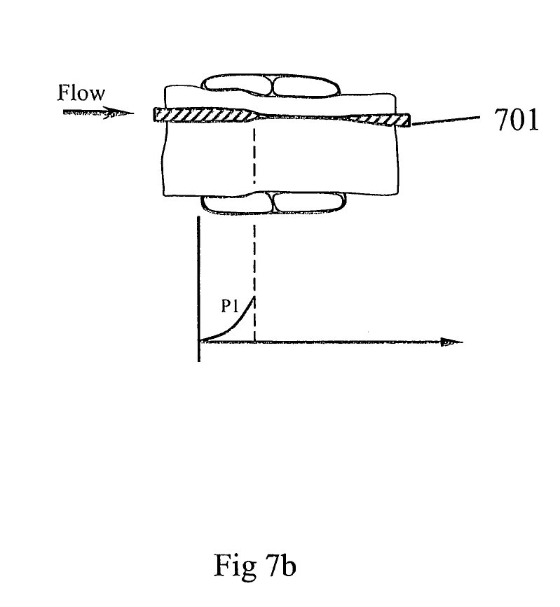

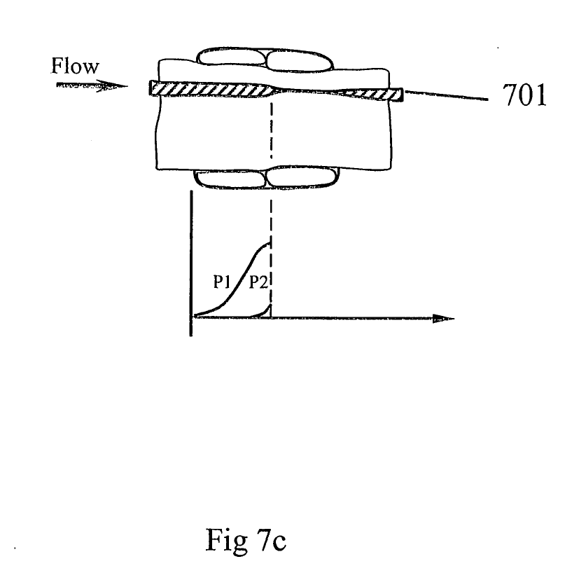

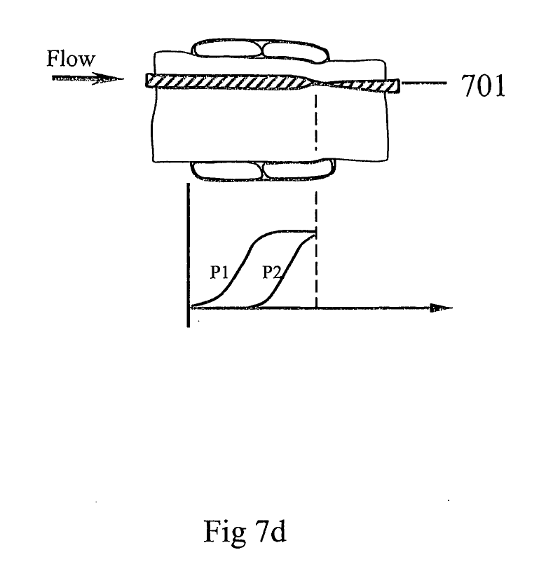

To better understand the algorithm for computing blood flow, consider the sequence of FIGS. 7a to 7e. The figures show the progress of the flow of blood through the artery as the blood is pushed forward during the heart cycle. This progress results in an increase of the cross section under the cuffs and consequent increase of the pressure within the cuff segments, which can be sensed by the sensors 503 and 504. The corresponding pressure profiles P1 and P2 are digitized by the A/D converter 601 and input to the micro-processor. The full cycle of pressure increase and decrease shown in FIG. 7 corresponds to a diastole to diastole cycle. An example of the measurements obtained by one sensor over the entire measurement is shown in FIG. 4, and in FIG. 8 after the quasi-static pressure of the cuff has been subtracted as described herein below. By quasi-static pressure is meant the pressure due to the inflatable device, which reduces gradually as the air exits the pneumatic circuit. Note that the signal from the more distal sensor is delayed with respect to the proximal sensor, by a quantity roughly proportional to the distance between the segments divided by the instantaneous velocity of the flow of blood.

While the quasi-static pressure in the cuffs is reduced from above systolic to below diastolic pressures, the pressure inside each of the segments of the sensor is measured and stored in memory. After all the pressure data has been collected, an algorithm is used to determine the pulse or set of pulses, corresponding each to one heart cycle, to be analyzed for the purpose of deducing hemodynamic parameters. The algorithm as applied in the embodiment of FIG. 3 comprises the following steps:

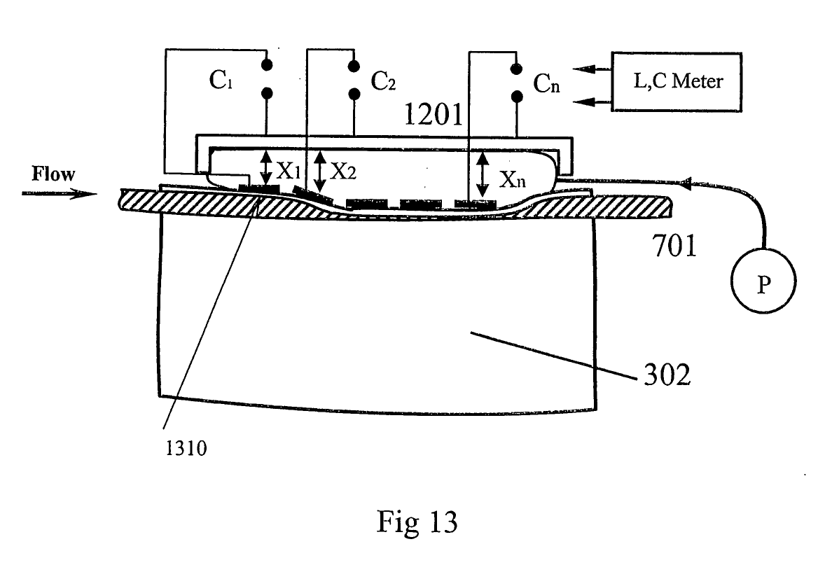

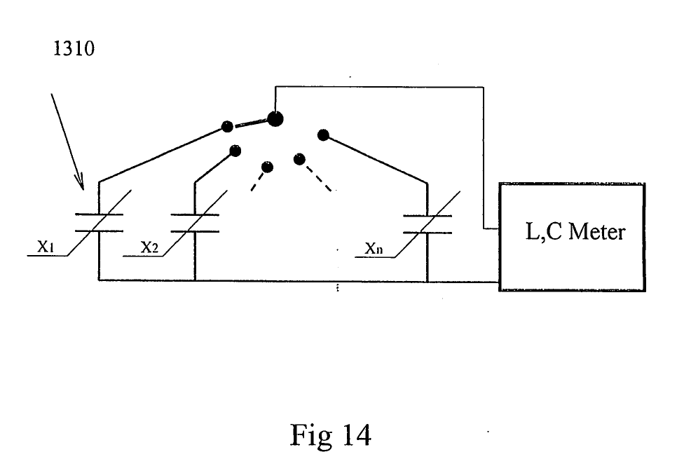

1) Calculating the instantaneous pressure changes within each segment of the cuff. This is carried out by subtracting the quasi static pressure from the pressure data of each segment. This can be done, for example by approximating the quasi-static pressure as a function of time using a low order polynomial fit to the pressure data, or by smoothing of the measurements data using appropriate low pass filter. The subtraction of the quasi-static pressure results in a time data containing instantaneous variations in internal pressure following the opening of the arteries. FIG. 14 shows a typical example of the resulting data.

2) Using the resulting nominal data history for one of the sensors the resulting nominal data history for one of the sensors (in the preferred embodiment, the proximal sensor), a search for the correct test pressure along the pressure curve is performed. Alternatively, the test pressure is determined by analyzing the variation of the quasi-static pressure in the cuff and using the algorithm known in the art for computing blood pressure. By test pressure is meant the quasi-static pressure at which the measurement data is analyzed to deduce the hemodynamic parameters of interest. Since different subjects have different diastolic and systolic blood pressures and same subject have different pressures at different times, it is desired to determined a test pressure that gives consistent results. In the preferred embodiment of the invention, consistent results are obtained by performing the measurement at the quasi pressure at which the modulation amplitude is the highest. Substantially, this is the mean pressure in the artery referred as MAP.

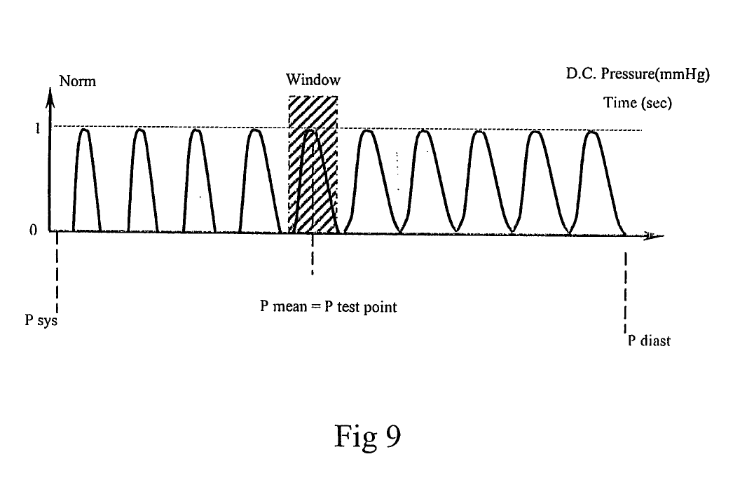

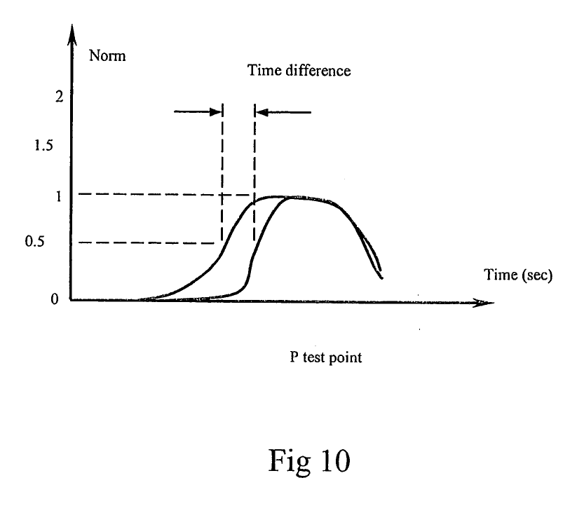

3) Once the test pressure and corresponding heart cycle have been determined, a time window is define to separate the data of one pulse as shown in FIG. 9. The pressure variations in that window for both cuff segments are process together as shown in FIG. 10.

4) The propagation time of the flow of blood from the proximal to the distal section is given by the time difference between the two curves shown in FIG. 16. Hence, the average propagation time is approximated by the average of the time difference between the curves above and below given thresholds. In the preferred embodiment of the invention, thresholds range of 0.2-0.8 are used for performing the calculations on clinical data.

5) The average velocity, called herein below as VP, is the distance between the cuff sections centers which are constant by construction of the apparatus, divided by the average propagation time computed as explained herein above.

6) In the preferred embodiment it is advantageous to analyze results for a number of pulses below and above the test pressure. Typically 3 to 5 pulses below and 2 to 3 pulses above are used. The pulse velocity value VP for the desired test pressure is determined by a polynomial fit of the velocity values above and below the test pressure. This procedure reduces sensitivity to noise and improves accuracy while still providing meaningful clinical results.

The detailed algorithm is provided herein above by a way of example. The reader experienced in the art will appreciate that other algorithms can be used to analyze the measurement data and extract the hemodynamics parameters within the scope of this invention.

In addition to the velocity VP, the apparatus described above measures the heart pulse rate HR and the systolic, mean and diastolic blood pressures using the pressure data and algorithms well known in the art.

Referring back to FIG. 6, in another preferred embodiment of the present invention the monitoring device and the analyzer are incorporated in a device worn by the patient on the wrist, this time with a double cuff sensor similar in operation to the design disclosed in FIG. 12c. As explained inhere above, for the wrist apparatus shown in FIG. 4 it is sufficient to focus the pressure on a small area adjacent to the radial artery, although it is also possible to use a device applying circumferential pressure. For understanding a preferred embodiment of a monitoring device mounted on the wrist reference is made to FIG. 12 showing that the artery on which the measurement is performed is preferably the radial artery. FIG. 11 shows the region 1101 on which the external pressure is applied on the wrist.

FIGS. 12a, 12b show a transversal cross-section of the wrist under this location. An arrow indicates the direction on which the blood flows. The figures do not show the strap or band required for applying external pressure as explained herein above. The strap or band is coupled to a mechanism, for example inflatable, for applying a external pressure. Alternatively the strap can be mechanically tightened, for example by means of a motorized rotor spinning strings about it thus shortening the strap, and reversing the direction of spin to loosen the strap. Other pressure applying straps can also be used.

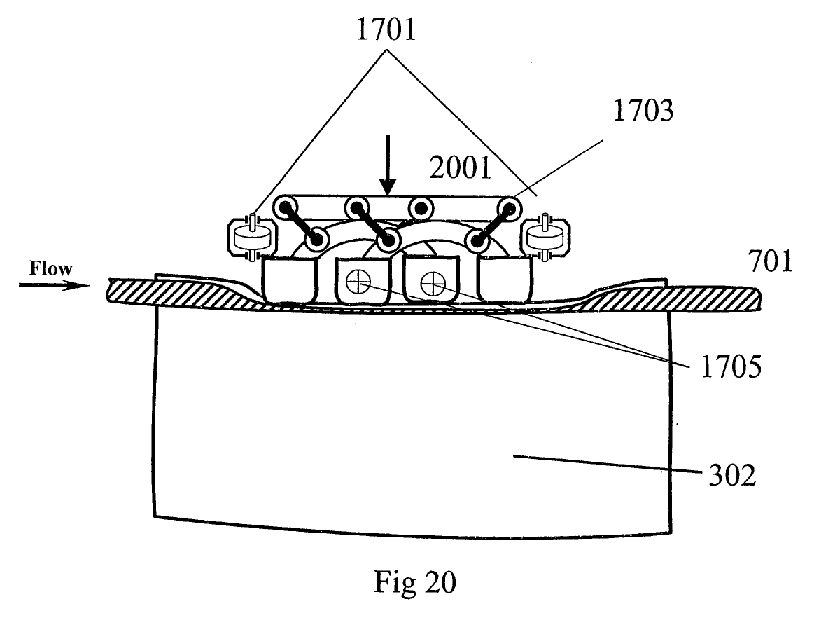

The apparatus applies an equally distributed force (pressure) on the region of interest (see FIG. 12a). The pressure is sufficiently high to produce the collapse of the artery, at least momentarily during the diastolic phase. This is illustrated in FIG. 20b, where the blood flow increases the internal pressure and opens the artery. It is important that the external pressure shall be equal at all points as can be achieved by means of a pneumatic device, similar to the cuff shown in FIG. 5, or by an other mechanical structures.

FIG. 12c shows a pressure applying structure of two cushions (1202, 1203) coupled to a bridge 1201, the cushions are inflatable as in the embodiment of FIG. 3, and provided with a pneumatic and control systems as shown in FIGS. 6 and 6. Bridge 1201 is secured to place by strap surrounding the wrist and provide counter-pressure when the cushions are inflated. The pneumatic and control system may be mounted in a separate unit or attached to the wrist mounted device providing a fully mobile battery operated device. The operation and data analysis of this device is identical to the embodiment described herein above for the upper arm.

The reader will appreciate that many mechanical arrangements can be used to apply the external pressure by reducing the circumference of the bracelet.

In an alternative preferred embodiment illustrated in FIG. 17, the mechanical changes due to the opening of the artery are sensed by applying pressure using cushions 1202 and 1203. These cushions are connected to a rigid bridge 1710 in such a way that pressure applied to bridge 1710 will be transmitted uniformly to cushions 1203, 1203. A solid state (or other type) gyroscope 1701 is mounted on the bridge for measuring the angular velocity about the pivot. The gyroscope 1701 may be, for example, the gyroscope marketed by MuRata™ under the name ENC-03G. The flex pivot 1711 is arranged so that the center of rotation of the pivot lies above the radial artery, to assure that a rotation motion of bridge 1710 will be created by the flow of blood.

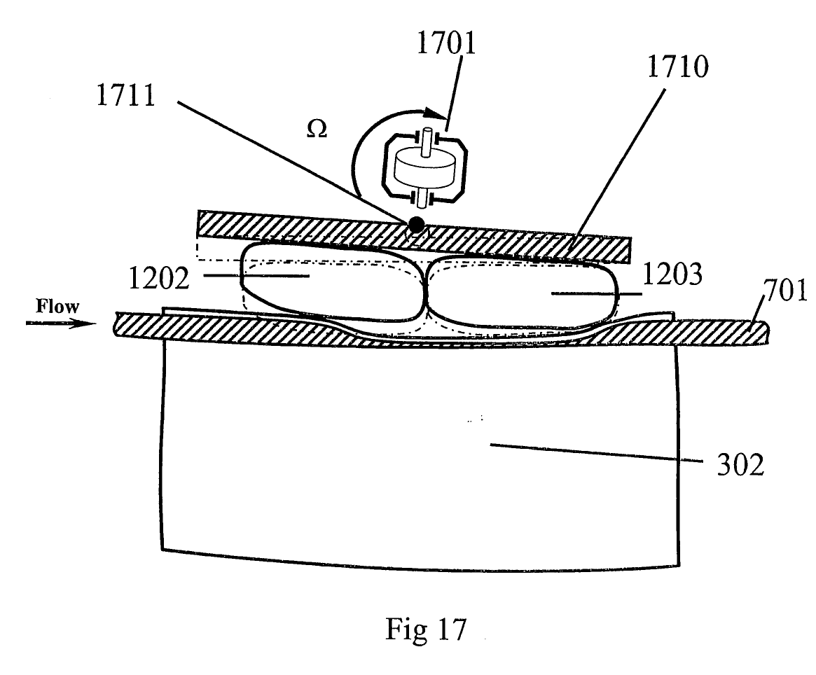

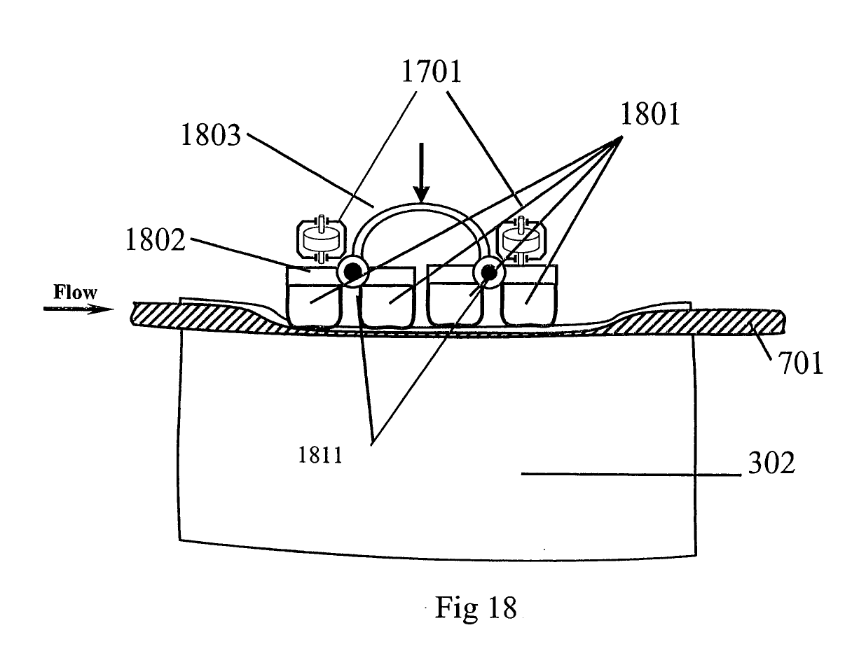

While a device with two cushions as shown in FIG. 17 generates good sensing signals, an alternative embodiment is preferred to increase sensitivity and accuracy of the measurement by using more than one pair of cushions as shown in FIG. 18. Cushions 1801 are coupled in pairs to plurality of bridges 1802, which are in turn connected to a top bridge 1803 that transmits external pressure uniformly to the lower side of the cushions and allows for rotational motion of the bridges 1802. The pressure is applied to top bridge 1803 by a mechanical arrangement. The joining device 1803 is attached to the bridges by means of bearings or by flex pivots 1811 to allow the appropriate transmission of the motion. The angular motion of sensors 1701 provides a measurement of the progress of the opening of the artery as explained inhere below.

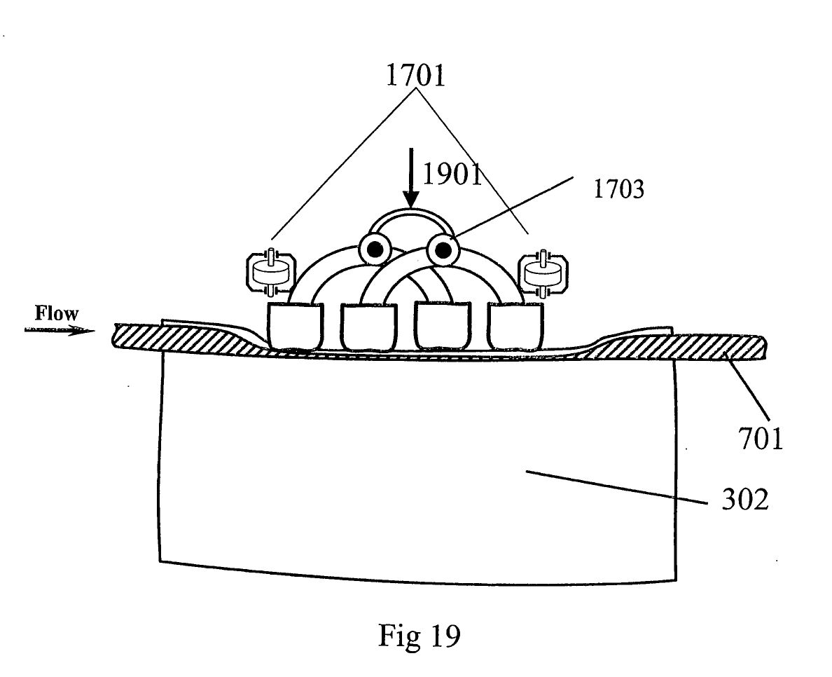

Two alternative embodiments of the same principle are shown in FIGS. 19 and 20. The functioning of these embodiment and the preceding embodiments using rigid cushions can be better understood by referring to FIG. 17. As explain herein above for the previous described embodiment, an external pressured is applied to collapse the artery of interest. The external pressure is than reduced monotonically as done in the previous embodiments to the region where the internal blood pressure is sufficient to open the blood vessel. The progress of the blood flow through the artery can be tracked. The unit volume of blood first raises the left leg of the bridge 1802 respective to FIG. 18 thus causing a clockwise rotation of the bridge. Then, as the blood flow progresses the bridge rotates in the opposite sense. The gyroscope 1701 senses these rotations. The A/D converter 601 samples the gyroscope measurements and inputs the digital signal into the microprocessor 602.

The analysis gyroscope output data in the embodiments shown in FIGS. 23-26 is described next. The microprocessor 602 transforms the rotational velocity into a measurement of the progress of the unit volume of blood through the artery, using the formulas:
V(t)≈(c+h/θ²)\dot{θ}, \dot{θ}≧0
V(t)≈−(c+h/θ²)\dot{θ}, \dot{θ}<0
In this formulas, V(t) represents the flow of blood velocity and \dot{θ} is the angular velocity of the bridge as measured by the gyroscope 1701. Plotting the results as a function of reducing pressure, a curve similar to FIG. 26 is obtained and is used to determine the desired test point. The microprocessor 602 determines the average wave velocity for that pulse.

The preferred embodiments described herein above are designed to follow the method of first increasing the external pressure above the desired test pressure and than gradually decreasing it while acquiring data. The test pressure at which the measurement is done is found from the acquired data and results are computed. It will be appreciated that the desired test pressure can also be found during gradual increase of the external pressure provided the means for generating the pressure do not interfere with the measurement. For example, if the external pressure is generated by inflating a cuff, the pump should provide smooth monotonic increase of the pressure and be shielded electronically from the sensors readout electronics. The advantage of such arrangement is that measurements can be taken more frequently by periodic increase and decrease of the pressure.

Embodiments based on pressure increase and decrease are most suitable for applications whereby the apparatus is programmed to repeat the measurement periodically at pre-set time intervals, or to provide a single measurement per user request. The control unit may be provided with means for the operators to program the measurement frequency and to initiate a single measurement.

However, any of the preferred embodiments herein above can be used, at least temporarily, also for continuous monitoring of hemodynamic parameters of interest. To this end, the external pressure has to be kept at approximately the optimal test pressure and acquisition of the sensor data is continuous. Under such conditions, the pulse velocity V_{P} can be computed for each pressure pulse and can be stored, processed and displayed as a function of time.

The following algorithm can be used for controlling the external pressure in embodiments for continuous monitoring of the blood flow:

1. Assuming the test pressure is the mean artery pressure, the test pressure is first found by overshooting it during monotonic pressure increase by observing the first pulses for which there is a reduction in the amplitude of subsequent pulses (see FIG. 14). The pressure is than reduced back to the desired test pressure.

2. The amplitude is calculated from pulse to pulse. If the amplitude is decreased, the external pressure is slightly decreased.

3. If the amplitude is increased the pressure is reduced further. If it is decreased, the pressure is increased till the amplitude starts to decrease.

4. Search for maximum amplitude continues till maximum reached.

5. No hemodynamic data is calculated for the short time intervals while the pressure is adjusted.

Notice that for those embodiments described herein above that apply pressure over the whole cross-section of the arm, a constant pressure can only be maintained for a few minutes since the flow of blood through the veins is interrupted.

The reader will appreciate that other procedures and algorithms can be applied as well to control the external pressure and are covered in the scope of the invention.

Any of the preferred embodiments inhere above can be provided with a memory unit to store the results of past measurement and later on display or transmit the patient past record. In particular, it is advantageous to store the results of the measurement for the patient while at rest in normal condition as a baseline to compare to further measurements during a condition of suspected decrease in cardiac output.

Furthermore, any of the preferred embodiments disclosed herein can be provided with means for transmitting the results of a recent measurement or the stored history data via telephone line, cellular telephone system, cord or cord-less communication line to a computer, direct link to computer network or any other mean of electronic communication.

Furthermore, any of the preferred embodiments inhere above can be provided with means to generate visible or audible alarm in case it identifies measurement results which indicated a possible situation of impaired heart performance. It is useful to store baseline normal condition data as a reference to detect abnormal results. The definition of alarming condition may be dependent not only on the velocity of the flow of blood but also on other parameters measured by the device such as heart rate and blood pressure.

Furthermore, any of the preferred embodiments inhere above can be integrated with other monitoring systems measuring other parameters to provide a complementary measurement. In some embodiments, the monitoring systems are ECG based monitors in hospital intensive care units. In other embodiments these are Holter systems used to monitor patients while they are carrying out their daily activity. The advantage of adding blood flow data to the ECG based monitoring is that false ECG alarms can be avoided by correlating the ECG with blood flow data.

It should be clear that the description of the embodiments and attached Figures set forth in this specification serves only for a better understanding of the invention, without limiting its scope.

It should also be clear that a person skilled in the art, after reading the present specification could make adjustments or amendments to the attached Figures and above described embodiments that would still be covered by the scope of the present invention.

1. A non-invasive apparatus for measuring cardiac mechanical performance of a patient, the apparatus comprising:
a pressure applying element mountable on a limb of the patient for applying pressure high enough to make a segment of an artery within the limb achieve a collapsed state and partially or totally empty it from blood at least momentarily;
at least one of a plurality of sensors coupled to said pressure applying element, sensing mechanical changes corresponding to volumetric changes in the artery as the artery progressively recuperates from its collapsed state;
processing unit communicating with said at least one of a plurality of sensors for receiving output corresponding to the mechanical changes from said at least one of a plurality of sensors and computing factors correlated with blood flow and calculate parameters indicating heart performance.

2. The apparatus as claimed in claim 1, wherein the pressure applying element is an inflatable cuff.

3. The apparatus as claimed in claim 1, wherein the pressure applying element is an inflatable cuff, divided into a plurality of inflatable segments.

4. The apparatus as claimed in claim 3, wherein the inflatable cuff is divided into at least two inflatable segments, and wherein said at least one of a plurality of sensors comprise at least two sensor transducers for detecting pressure changes within the segment, each transducer corresponding to a different segment.

5. The apparatus as claimed in claim 3, wherein the pressure applying element is operated by a pneumatic system comprising a pump for increasing the pressure within the cuff, and valves for releasing the pressure from the cuff.

6. The apparatus as claimed in claim 1, wherein the pressure applying element is driven by an electrical motor.

7. The apparatus as claimed in claim 1, wherein the pressure applying element is coupled to a bracelet having a diameter which is automatically adjustable.

8. The apparatus as claimed in claim 7, wherein the bracelet consists of a strap and wherein bracelet's diameter may be increased or decreased by turning a screw operated by a motor to which the strap is attached.

9. The apparatus as claimed in claim 7, wherein the pressure applying element is hydraulically operated.

10. The apparatus as claimed in claim 1 wherein the pressure applying element comprises said at least one of the plurality of cushions held against the limb by a rigid bridge.

11. The apparatus as claimed in claim 10, wherein the cushions are inflatable.

12. The apparatus as claimed in claim 1, wherein the pressure applying element comprises at least one of a plurality of cushions held against the limb by a rigid bridge, and wherein said at least one of a plurality of sensors comprises deformation sensors, sensing deformation changes of said at least one of the plurality of cushions.

13. The apparatus as claimed in claim 12, wherein said at least one of the plurality of cushions is inflatable.

14. The apparatus as claimed in claim 12, wherein said at least one of the plurality of cushions is filled with hydraulic fluid.

15. The apparatus as claimed in claim 12, wherein the deformation sensors comprise an array of capacitors, wherein the mechanical changes are determined by measuring changes in the capacitance of the capacitors, due to deformation changes.

16. The apparatus as claimed in claim 1 wherein said at least one of a plurality of sensors include an array of piezoelectric transducers wherein the mechanical changes are determined by measuring changes in the output voltage of the transducers.

17. The apparatus as claimed in claim 1 wherein said at least one of a plurality of sensors include an array of conducting rubber transducers wherein the mechanical changes are determined by measuring changes in the resistance of the said conductive rubber strips.

18. The apparatus as claimed in claim 1, wherein the pressure applying element comprises at least one cushion held against the limb by at least one of a plurality of pivotal rigid bridges, each provided with gyroscopic sensor to sense rotational velocity of said at least one of a plurality of pivotal rigid bridges.

19. The apparatus as claimed in claim 18, wherein said at least one of a plurality of pivotal rigid bridges comprise two pivotal bridges.

20. The apparatus as claimed in claim 19, wherein the two pivotal bridges are coupled to a third pivotal bridge.

21. The apparatus as claimed in claim 1, further comprising output means.

22. The apparatus as claimed in claim 1, further comprising memory unit.

23. The apparatus as claimed in claim 1, further comprising means to communicate with a computer, network or a telephone system.

24. The apparatus as claimed in claims 1 or 17, wherein the processing unit includes algorithm comprising the following steps:
a. identification of piezoelectric output pulses with magnitude above certain threshold where at least a pre-determined number of pulses fall within a pre-determined time window;
b. determining time differences between the pulses corresponding to same time window;
c. determining average propagation of the flow of blood from known piezoelectric elements positions and timing relative to each other.

25. The apparatus as claimed in claim 1 wherein a control system is used to maintain the applied pressure over a period of time substantially at the a determined measurement pressure and factors correlated with pulse wave propagation are measured continuously.

26. The apparatus as claimed in claim 17, wherein the velocity of propagation of the flow of blood is calculated from the combined data of plurality of sensors, each detecting pressure changes at corresponding segment of the patient's limb.

27. The apparatus as claimed in claim 17, wherein the propagation of the flow of blood velocity is calculated from the time difference between data of plurality of sensors, each detecting pressure changes.

28. The apparatus as claimed in claim 17, wherein the propagation of the flow of blood velocity is calculated by a fit of a theoretical curve to data indicating sensor segment triggering time versus said segment position.

29. The apparatus as claimed in claim 17, wherein the diastolic and systolic blood pressures of the subject are determined from the piezoelectric output signals.

30. The apparatus as claimed in claim 17, wherein the diastolic blood pressure is determined as a value at or below the lowest pressure at which there is at least one of piezoelectric output signal satisfying pre-determined conditions and the systolic blood pressure is identified as a value at or above the highest pressure at which there is at least one of piezoelectric output signal satisfying pre-determined conditions.

31. The apparatus as claimed in claim 17 wherein the heart rate is determined from piezoelectric output signals.

32. The apparatus as claimed in claims 1 or 17, wherein the processing unit includes algorithm comprising the following steps:
a. calculating instantaneous pressure changes within the pressure inducing member as a function of time;
b. dividing the instantaneous pressure changes into segments corresponding to pulse rate periods of the patient;
c. finding the mean artery pressure and analyzing at least one segment located within 5 pulse rates from the mean artery pressure.

33. The apparatus as claimed in claims 1 or 17, wherein the mean artery pressure is found by gradually increasing the applied pressure while acquiring pressure data.

34. The apparatus as claimed in claims 1 or 17, wherein a control system is used to maintain the applied pressure over a period of time substantially at the mean artery pressure and factors correlated with pulse wave propagation are measured continuously.

35. The apparatus as claimed in claims 1 or 17 wherein the measurement data is used to calculate the velocity of propagation of the flow of blood.

36. The apparatus as claimed in claim 3, wherein the velocity of propagation of the flow of blood is calculated from the combined data of plurality of sensors, each detecting pressure changes within corresponding segment of the inflatable cuff.

37. The apparatus as claimed in claim 3, wherein the velocity of propagation of the flow of blood is calculated from the time difference between data of plurality of sensors, each detecting pressure changes within corresponding segment of the inflatable cuff.

38. The apparatus as claimed in claim 3, wherein the velocity of propagation of the flow of blood is calculated by a fit of a theoretical curve to data indicating sensor segment triggering time versus said segment position.

39. A method for non-invasive measuring of changes in cardiac mechanical performance of a patient, the method comprising:
a. providing a pressure applying element mountable on a limb of the patient for applying pressure enough to make a longitudinal segment of an artery within the limb achieve a collapsed state and empty it from blood at least momentarily;
b. providing sensor coupled to the pressure applying element, sensing mechanical changes corresponding to volumetric changes in the artery as the artery progressively recuperates from its collapsed state;
c. providing processing unit communicating with the sensor for receiving output corresponding to the mechanical changes from the sensor and computing factors correlated with blood flow and calculate parameters indicating heart performance;
d. applying pressure on a portion a limb of a patient through which artery passes enough to collapse the artery preventing at least momentarily the flow of blood through the collapsed artery;
e. sensing mechanical changes corresponding to volumetric changes in the artery as the artery progressively recuperates from its collapsed state;
f. computing factors correlated with progression of artery recuperation and calculating parameters indicating heart performance.

40. The method as claimed in claim 39, wherein the pressure applied on the portion of the limb of the patient is initially larger than needed to collapse the artery, and wherein it is gradually reduced, sensing the mechanical changes correlating to the volumetric changes while the pressure is reduced.

41. The method as claimed in claim 39, further comprising determining a best cuff pressure for considering a measurement, said best pressure is the mean artery pressure or other pressure pre-determined relative to the diastolic and systolic blood pressures.

42. The method as claimed in claim 39, further comprising measuring blood pressure of the patient.

43. The method as claimed in claim 39, further comprising measuring heart pulse rate of the patient.

44. The method as claimed in claim 39, carried out continuously over a period of time.

45. The method as claimed in claim 39, further comprising transmitting data to an external apparatus.

46. The method as claimed in claim 33, wherein it is incorporated with Holter procedure, in order to detect artifacts and enhance reliability.
